(19) United States
(12) Patent Application Publication
(10) Pub. No.: US 2009/0156459 A1
(43) Pub. Date: Jun. 18, 2009

(54) Cationic-Core Carrier Compositions for Delivery of Therapeutic Agents, Methods of Making and Using the Same

(75) Inventors: Gerardo M. Castillo, Bothell, WA (US); Elijah M. Bolotin, Bothell, WA (US)

Correspondence Address: WILSON SONSINI GOODRICH AND ROSATI / PHARMAIN LTD, 650 PAGE MILL ROAD, PALO ALTO, CA, 94304, US

(73) Assignee: PharmaIN Corporation, Seattle, WA (US)

(21) Appl. No.: 12/271,732

(22) Filed: Nov. 14, 2008

Related U.S. Application Data
(60) Provisional application No. 60/988,669, filed on Nov. 16, 2007.

(51) Int. Cl. A61K 38/02; C07K 2/00

(52) U.S. Cl. 514/2; 530/300

(57) ABSTRACT

The present invention relates to a biocompatible cationic-core carrier composition that has sustained release capability and includes a polymeric backbone, protective chains, poly-cationic moieties and optionally an anionic load molecule.

### CROSS-REFERENCE

This application claims the benefit of U.S. Provisional Application No. 60/988,669 filed Nov. 16, 2007, which application is incorporated herein by reference in its entirety.

### BACKGROUND OF THE INVENTION

The development of new formulations and delivery systems for administration of physiologically active oligonucleotides, DNA, RNA, negatively charged peptides, negatively charged proteins, and other anionic drugs or therapeutics is driven by the need to achieve the desirable physiological effects. Oligonucleotides, DNA, RNA, peptides, and proteins have been observed to be unstable in the blood and the gastrointestinal tract. In addition, those that have low molecular masses tend to have short biological half-lives due to their removal from systemic circulation via the kidneys. Furthermore, a fraction of them can also be removed via reticuloendothelial uptake due to recognition by monocyte/macrophages or as a result of opsonization by complement components. They can also lose their activity in vivo due to nucleases (RNAses and DNAses) and proteases. They may need to be stabilized or protected prior to delivery and remain protected in the circulation and gastro-intestinal tract or the circulation following delivery.

It has been over a decade since oligonucleotides such as siRNA and antisense RNA and DNA were discovered. However their therapeutic potential remains unrealized due to their rapid degradation and instability in vivo. This is also true for therapeutic peptides and proteins. For oligonucleotides to be effective in inhibiting translation of specific genes, large doses are required which often induce toxicity to the organism being treated. The toxicity is not related to inhibition of translation of target genes per se but due to the overwhelming amount of materials being used (over 10 mg/kg). There is a long felt need to stabilize oligonucleotides in biological fluid to realize the promised potential of oligonucleotide therapies. An approach that had been tried includes complexation of oligonucleotide with cationic polymer and allowing the whole complex (nucleotide and cationic polymer) to be internalized by cells. No approaches that have been presented to date that will allow an oligonucleotide to circulate in the blood in a protected reservoir, where the oligonucleotide is bound to a carrier which can release oligonucleotide in a sustained manner. The released oligonucleotide can then be internalized by the cells independent of the carrier. There exists a need for a sustained release oligonucleotide delivery system that works for a wide range of oligonucleotides and where the release rate is readily controlled.

Advances in nucleic acid technology allow manufacture of the active ingredient in gene-based medicines, such as the gene itself or its corresponding RNA in many forms including siRNA. However the delivery of these genetic materials (DNA, their corresponding RNAs or siRNAs) to sites of pathology still remains a major hurdle. Viral gene delivery vectors have been tested and found to give stable expression in the case of adenoviruses. However, adenoviruses can result in a severe immunological reaction that precludes administration of a repeat dose of the gene. Retroviruses on the other hand which, hold the advantage of preferentially infecting actively dividing cells, are more likely to insert DNA in the host genome with unknown consequences.

Polylysine has previously been modified by the attachment of phospholipid groups and used in DNA transfection (Zhou X H et al (1991) Biochim. Biophys. Acta 1065: 8-14 and Zhou X H, Huang L (1994) Biochim. Biophys Acta 1189: 195-203). Polylysine has also been previously modified by the attachment of hydrophilic groups such as polyethylene glycol (Dash P R, et al (1997) J. Contr. Rel. 48: 269-276 and Toncheva V, et al (1998) Biochim Biophys Acta. 1380:354-368) and various sugars (Kollen W J W, et al, (1996) Human Gene Ther. 13: 1577-1586 and Erbacher P, et al (1997) Biochim Biophys Acta 1324: 27-36). However, although polylysine can act as poly-cationic group that binds nucleotide, the binding is very tight as reported since the majority of the groups were not derivatized with PEG.

There exists a medical need for the development of solutions for the delivery of medicinally active load molecules.

### SUMMARY OF THE INVENTION

It is an object of the present invention to provide a delivery system for therapeutic agent that has sustained release capability, safe, biocompatible, readily prepared from known chemistries and compounds, amenable to a wide variety of therapeutic agents, and where the release rate can be readily adjusted by simple mechanisms of altering the poly-cationic core characteristics of the delivery system.

The instant application discloses a biocompatible composition comprising of a polymeric carrier that is modified so as to bear multiple hydrophilic protective groups such as PEG, PEG derivatives or PEG substitute and at least one poly-cationic moiety to which the anionic therapeutics such as RNAs, DNAs, peptides, proteins, and other drugs can reversibly bind with affinity (Ka) of greater than 0.1 million/M or a dissociation constant (Kd) of 10 micromolar or less. The carrier can reversibly bind anionic load molecules such as oligonucleotides, RNA, DNA, proteins, peptides, and anionic drugs or therapeutics. The association constant (Ka) or dissociation constant (Kd) between the carrier and load molecules will depend on the number and density of each poly-cationic sites in the carrier and the number and density of anionic sites in the load molecules. The concentration of free anionic load molecules not associated with the carrier and the further release of anionic load molecules from the carrier when the concentration of free anionic load molecules goes down is the result of the desire of the system to achieve the equilibrium constant (Ka or Kd). In essence, this is an affinity based carrier which uses the equilibrium constant to control the level of drug in the system which distinguishes the carrier of the present invention from system that relies on slow rate of release due physical degradation of the carrier.

In the present invention, the amount of a protective group such as MPEG in the polymeric backbone prior to addition of poly-cation (such as branched polyethyleneimine) is relatively high such that the binding of oligonucleotide is of low affinity (with an affinity constant (Ka) of less than 0.1 million/molar or dissociation constant (Kd=1/Ka) of greater than 10 uM). The compositions described here restore the anionic charge by covalently linking more poly-cation (such as branched polyethyleneimine) to the remaining amino residues of the polymer. This process restores the ability of the polymer to bind polynucleotide with sufficiently high affinity while having sufficient density of the protective group to protect the poly-cation from elimination in vivo. The number of polyethylene glycol protective chains is high enough to protect the carrier and there is sufficient number and density of poly-cation to provide high capacity and high affinity interaction with anionic molecules (described in examples). It was also observed that the structures of the present invention do not form supramolecular structures (a structure with a hydrated molecular diameter of 70 to 200 nm or greater) when bound to oligonucleotide making the present invention novel. The polymer of the present invention is designed such that the size of each poly-cationic group attached to the polymeric residue is less than one fourth of a protective group providing sufficient protection for each cationic-anionic load molecule complex.

The present invention relates to a polymeric composition formed from at least three polymers wherein two polymers (a protective group and a poly-cationic moiety) are pendants to one linear polymeric backbone polymer. The polymeric backbone is modified so as to bear multiple hydrophilic protective groups of at least 2 kDa but no more than 20 kDa and at least one poly-cationic moiety of no more than 25% of the molecular weight of individual or average protective groups. The compositions are suited for prolonging the blood circulation half-life of anionic molecules such as RNA, DNA, anionic proteins, anionic peptides, and anionic drugs or therapeutics that are associated with the poly-cationic moiety of the composition. The compositions are suited for stabilizing and reducing the rate of breakdown of anionic molecules such as RNA, DNA, proteins, peptides, and anionic drugs or therapeutics that are associated with the poly-cationic portion of the composition.

In the present invention, the amount of protective group MPEG in the polylysine prior to addition of poly-cation (such as branched polyethyleneimine) is so high that the binding of oligonucleotide is of low affinity (with an affinity constant (Ka) of less than 0.1 million/molar or dissociation constant (Kd=1/Ka) of greater than 10 uM). So, our starting polylysine-PEG conjugate is not identical to the above references, further we restore the anionic charge by covalently linking more poly-cation (such as branched polyethyleneimine) to the remaining amino residues of lysine. This process restored the ability of the polymer to bind polynucleotide with sufficiently high affinity while having sufficient density of protective group to protect the poly-cation from rapid elimination in vivo. The number of polyethylene glycol protective chains is high enough to protect the carrier and there is sufficient number and density of poly-cation to provide high capacity and high affinity interaction with anionic molecules (see results). We also discovered that this structure of the present invention does not form supramolecular structure (a structure with hydrated molecular diameter of 70 to 200 nm or greater) when bound to oligonucleotide making the present invention distinct from existing art. The polymer of the present invention is designed such that the size of each poly-cationic group attached to the polylysine residue is less than one fourth of single protective group providing sufficient protection for each cationic-anionic load molecule complex. In addition various drugs (Hudecz F. et al (1993) Bioconjugate chemistry 4: 25-33) and targeting residues such as transferrin (Wagner, E (1994) Adv. Drug Delivery Rev. 14: 113-135), asialoglycoprotein (Chowdhury, N R et al (1993) J. Biol. Chem. 268: 11265-11271) and monoclonal antibodies (Chen, J B et al (1994) Febs Lett 338: 167-169) have been conjugated to polylysine. None of these have a poly-cationic groups, other than poly-lysine backbone, attached to the backbone and protective groups directly attached to lysine residues such that the size of each poly-cationic group is less than one fourth of single protective group.

In this context, one of the objects of the present invention is to provide a novel protected graft co-polymeric carrier with a backbone made up of repeating units with modifiable functional groups (such as amino, carboxyl, hydroxyl, sulfur, and phosphate) modified to contain poly-cationic groups and protective groups pendant to the polymeric backbone such that the molecular weight of each poly-cationic group is less than one fourth of a single protective group.

Embodiments of the present invention pertain to carriers which are derivatized polymer backbones of at least 30 residues with covalently linked hydrophilic protective groups of at least 2000 kDa are each pendant to the polymer backbone, and poly-cationic groups independently linked and pendant to the polymer backbone. The molecular weight of each poly-cationic group is less than one fourth of the average molecular weight of hydrophilic protective groups. The size difference between the hydrophilic protective group and the poly-cationic group is essential to protect the anionic load molecules from proteases and nucleases. This also protects the entire carrier with anionic load molecules from the reticuloendothelial system.

In a further embodiment of the present invention, the sustained release delivery system may optionally include a targeting moiety for efficient delivery of the therapeutic agent to a site in need thereof.

It is another object of the present invention to provide a method of treating a disorder by delivering a therapeutic agent to a patient in need thereof in a controlled manner and at a release rate that is safe and effective and readily adjusted to be so.

The subject invention results from the long felt need to deliver polynucleotides such as siRNA, microRNA, anionic peptides, anionic proteins and anionic drugs in patients as therapeutic molecules in a controlled manner. Controlled manner means that the level of the active therapeutic molecules in the circulation will not exceed a toxic level and will not go below the therapeutically effective level for the desired period of time. The ability of the carrier of the present invention to release free and active therapeutic agent, or in a broader sense, a load molecule, when the level of free load molecule in the circulation goes below the therapeutically effective level may be readily adjusted. The carriers of the present invention are safe and non-immunogenic. The carriers of the present invention may be prepared to have both high loading capacity and adjustable release rates by controlling the number of positive charges in the poly-cationic moiety and optionally the associated hydrophobic group of the poly-cationic moiety.

In part, the present invention is directed towards novel drug delivery systems or imaging agents, and methods of making and using the same.

The present invention relates to a biocompatible poly-cationic core carrier composition comprising: (i) a linear polymeric backbone, wherein the backbone may be polylysine, polyaspartic acid, polyglutarnic acid, polyserine, polythreonine, polycysteine, polyglycerol, polyethyleneimines, natural saccharides, aminated polysaccharides, aminated oligosaccharides, polyamidoamine, polyacrylic acids, polyalcohols, sulfonated polysaccharides, sulfonated oligosaccharides, carboxylated polysaccharides, carboxylated oligosaccharides, aminocarboxylated polysaccharides, aminocarboxylated oligosaccharides, carboxymethylated polysaccharides, or carboxymethylated oligosaccharides; (ii) a plurality of polymeric protective chains covalently linked and pendant to the polymeric backbone, wherein each protective side chain has a molecular weight between about 400 Daltons and 20,000 Daltons and the protective chain comprises of polyethylene glycol, polypropylene glycol, a co-polymer of polyethylene glycol and polypropylene glycol, polysaccharide, or alkoxy derivatives thereof, (iii) a poly-cationic moiety covalently linked and pendant to the polymeric backbone with molecular weight of less than 25% of the molecular weight of each protective chain, wherein poly-cationic moiety may be polyamine such as polyethyleneimine, putrescine, spermine, spermidine, cadaverine, polylysine, polyarginine or derivative thereof, and optionally (iv) a load molecule dissociably linked to the Poly-cationic moiety.

In various embodiments, the load molecule may be a therapeutic agent or an imaging agent. In one embodiment, the therapeutic agent may be a polynucleotide, anionic peptide, anionic protein, or anionic drugs. The polynucleotide may be RNA, DNA or derivatives thereof. The polynucleotide may be oligonucleotide of RNA, DNA or derivatives thereof. The RNA may be siRNA, microRNA, or anti-sense RNA. The DNA may be anti-sense DNA. The anionic peptide/protein may or may not be oligonucleotide-bonded peptide/proteins; wherein the peptide/protein may be peptide aptamer, glucagon-like-peptide, glucagon-like-peptide derivative, exenatide, leptin, leptin fragment, Peptide YY, alpha-melanocyte stimulating hormone, adiponectin, Gastric inhibitory polypeptide(GIP), Epidermal Growth Factor (EGF) receptor ligand, EGF, Transforming Growth Factor alpha (TGF-alpha), Betacellulin, Gastrin/Cholecystokinin receptor ligand, Gastrin, Cholecystokinin, lysostaphin, interferon, interferon gamma, interferon beta, interferon alpha, interleukin-1, interleukin-2, interleukin-4, interleukin-6, interleukin-8, interleukin-10, interleukin-12, tumor necrosis factor, tumor necrosis factor alpha, tumor necrosis factor beta, insulin, insulin-like growth factor, growth hormone, nerve growth factor, brain-derived neurotrophic factor, enzymes, endostatin, angiostatin, thrombospondin, urokinase, streptokinase, blood clotting factor VII, blood clotting factor VIII, granulocyte-macrophage colony-stimulating factor (GM-CSF), granulocyte colony-stimulating factor (G-CSF), thrombopoietin, calcitonin, parathyroid hormone (PTH) and its fragments, erythropoictin, atrial natriuretic factor, monoclonal antibodies, monoclonal antibody fragments, somatostatin, protease inhibitors, adrenocorticotropin, gonadotropin releasing hormone, oxytocin, leutinizing-hormone-releasing-hormone, follicle stimulating hormone, glucocerebrosidase, thrombopoietin, filgrastin, terlipressin, and vasoactive intestinal peptide (VIP). The siRNA load molecule may against EGR1 gene, SSB gene, Ghrelin, NPY, Cathepsin, Myostatin, TSLP, IL-4, IL-8, L-12, IL-13, STAT-6, MIP-1 alpha, RANTES, CCR1, CCR3, INF-gamma, TNF-alpha, ICXCR1, MCP-1, and CCR2, CXCR3, CXCR4, CXCR5, CCR4, CCR5, CCR6, CCR7, CCR8, gp120, gp41, p17, p24, RT, HIV proteases, Fas (CD95), FAS-L, FADD, Caspase-8, IL-1, IL-6, Bak, flax, Bid, Bcl-2, Bcl-XL, HLA-G, IGF-1, EGF, FGF, VEGF, VEGFR, IGFR, EGFR, FGFR, HER2, TGF-beta, Caspase 3, CEACAM6, HPV-E6, HPV-E7, H-Ras gene, P100a gene, CREB, BRAF gene, ATF2, N-myc gene, Cox1, Cox2, GluR2, DAT, VEGFR1, TGF-b-RII, IL-1-beta, Facipain-1-2 malaria protein, viral Capsid protein, NS5A, NP influenza protein, PA influenza protein, and HBV.

In a further embodiment the present invention relates to any of the aforementioned compositions, further comprising of hydrophobic group covalently linked to the poly-cationic moiety. In a one embodiment, the hydrophobic group comprises alkyl group. In one embodiment, the alkyl group comprises an un-branched alkyl group. In another embodiment, the un-branched alkyl group comprises a double bond. In another embodiment, the alkyl group comprises a branched alkyl group. In another embodiment, the branched alkyl group comprises a double bond. In another embodiment, the alkyl group comprises a methyl, ethyl or propyl group. In another embodiment the alkyl group is a butyl, pentyl or hexyl group. In another embodiment, the alkyl group is CH₃(CH₂)ₙCO—, —OC(CH₂)ₙCH₂—, —OC(CH₂)ₙCH₂ NH—, —OC(CH₂)ₙCO—, —OC(CH₂)ₙCH₂O—, —OC(CH₂)ₙCH₂S—, —HNC(CH₂)ₙCH₂—, —HNC(CH₂)ₙCO—, —OCH₂(CH₂)ₙCH₂—, or —OCH₂ (CH₂)ₙCO—; wherein “n” is 4-36, inclusive. In another embodiment, the hydrophobic groups comprise an aromatic ring compound. In another embodiment, the aromatic ring is phenyl. In another embodiment, the aromatic ring is naphthyl. In a further embodiment, the aromatic ring compound is cholesterol.

In further embodiment the present invention relates to the aforementioned compositions without hydrophobic groups further comprising of second protective chains covalently linked to the poly-cationic moiety in addition to the first protective chains covalently linked to the polymeric backbone. In another embodiment the present invention relates to the aforementioned compositions with hydrophobic group further comprising of second protective chains covalently linked to the hydrophobic group in addition to the first protective chains covalently linked to the polymeric backbone.

In further embodiment the present invention relates to all the aforementioned compositions further comprising of targeting moiety covalently linked to the distal end the protective group. The targeting moiety may be an antibody, fragment of an antibody, chimeric antibody, lectins, receptor ligands, proteins, enzymes, peptides, saccharides, quasi substrates of enzymes, cell-surface-binding compounds, and extracellular-matrix-binding compounds.

In one aspect, the invention provides a cationic-core carrier composition comprising a polymeric backbone, a plurality of polymeric protective chains covalently linked and pendant to the polymeric backbone and a plurality of poly-cationic moieties covalently linked and pendant to the polymeric backbone. In a related aspect, each poly-cationic moiety covalently linked and pendant to the polymeric backbone has a molecular weight of no more than 25% of the average molecular weight of the protective chains, wherein each protective side chain has a molecular weight between about 400 and 20,000 Daltons. In one embodiment, the polymeric backbone is linear. In another embodiment a load molecule dissociably linked to the poly-cationic moiety. In some embodiments the load molecule is a therapeutic agent and the agent is selected from the group consisting of a polynucleotide, an anionic peptide, an anionic protein, an anionic drug, and an oligonucleotide covalently bonded to a peptide or protein. In a certain embodiment the therapeutic agent is RNA, siRNA, or DNA. In specific embodiments the siRNA is against any one from the group consisting of SSB gene, Ghrelin, NPY, Cathepsin, Myostatin, TSLP, IL-4, IL-8, L-12, IL-13, STAT-6, MIP-1 alpha, RANTES, CCR1, CCR3, INF-gamma, TNF-alpha, ICXCR1, MCP-1, and CCR2, CXCR3, CXCR4, CXCR5, CCR4, CCR5, CCR6, CCR7, CCR8, gp120, gp41, p17, p24, RT, HIV proteases, Fas (CD95), FAS-L, FADD, Caspase-8, IL-1, IL-6, Bak, Bax, Bid, Bcl-2, Bcl-XL, HLA-G, IGF-1, EGF, FGF, VEGF, VEGFR, IGFR, EGFR, FGFR, HER2, TGF-beta, Caspase 3, CEACAM6, HPV-E6, HPV-E7, H-Ras gene, P100a gene, CREB, BRAF gene, ATF2, N-myc gene, Cox1, Cox2, GluR2, DAT, VEGFR1, TGF-b-RII, IL-1-beta, Facipain-1-2 malaria protein, viral Capsid protein, NS5A, NP influenza protein, PA influenza protein, and HBV. In some embodiments the backbone is selected from the group consisting of polylysine, polyaspartic acid, polyglutamic acid, polyserine, polythreonine, polycysteine, polyglycerol, polyhistidine, natural saccharides, aminated polysaccharides, aminated oligosaccharides, polyamidoamine, polyacrylic acids, polyalcohols, sulfonated polysaccharides, sulfonated oligosaccharides, carboxylated polysaccharides, carboxylated oligosaccharides, aminocarboxylated polysaccharides, aminocarboxylated oligosaccharides, carboxymethylated polysaccharides, and carboxymethylated oligosaccharides. In specific embodiments the polymeric backbone is polylysine. In some embodiments the protective side chain is polyethylene glycol, polypropylene glycol, a co-polymer of polyethylene glycol and polypropylene glycol, polysaccharide, or alkoxy derivatives thereof. In further embodiments the alkoxy derivative is methoxypolyethylene glycol, methoxypolypropylene glycol, a methoxylated co-polymer polyethylene glycol and polypropyleneglycol, or ethoxylated polysaccharide. In specific embodiments the protective side chain is methoxypolyethylene glycol. In some embodiments the poly-cationic molecule is selected from polyethyleneimine, spermidine, spermine, putrescine, cadaverine, polylysine, poly-arginine, and derivatives thereof. In specific embodiments the poly-cationic moiety is polyethyleneimine. In a specific embodiment the poly-cationic moiety is polyethyleneimine. In some further embodiments a targeting molecule is covalently linked to the protective side chains. In specific embodiments the targeting molecule is selected from a group consisting of an antibody, fragment of an antibody, chimeric antibody, lectins, receptor ligands, proteins, enzymes, peptides, saccharides, quasi substrates of enzymes, cell-surface-binding compounds, and extracellular-matrix-binding compounds. In some embodiments the composition further comprises hydrophobic groups covalently linked to the poly-cationic moiety, wherein each hydrophobic group has molecular weight of 15-700 Da, independent of the poly-cationic moiety. In specific embodiments the hydrophobic group is an alkyl group with 1 to 36 carbon atoms. In some embodiments the composition further comprises a second set of protective chains covalently linked to the hydrophobic group or to the poly-cationic moiety. In a related embodiment, the invention provides for a pharmaceutical composition.

In another aspect the invention provides a cationic-core carrier composition comprising a polymeric backbone, a plurality of polymeric protective chains covalently linked and pendant to the polymeric backbone, a plurality of poly-cationic moieties covalently linked and pendant to the polymeric backbone, each with molecular weight of no more than 25% of the average molecular weight of the protective chains, wherein each protective side chain has a molecular weight between about 400 and 20,000 Daltons, a load molecule dissociably linked to the poly-cationic moiety, and hydrophobic groups covalently linked to the poly-cationic moiety, wherein each hydrophobic group has molecular weight of 15-700 Da, independent of the poly-cationic moiety. In a related embodiment the hydrophobic group is an alkyl group with 1 to 36 carbon atoms. In a related composition there is a second set of protective chains covalently linked to the hydrophobic group. In any of these embodiments a load molecule such as prostaglandin can be dissociably linked. In a related embodiment, the invention provides for a pharmaceutical composition.

In another aspect the invention provides a cationic-core carrier composition comprising a polylysine backbone, a plurality of polymeric protective chains covalently linked and pendant to the polylysine backbone, a plurality of poly-cationic moieties covalently linked and pendant to the polylysine backbone, each with molecular weight of no more than 25% of the average molecular weight of the protective chains, wherein each protective side chain has a molecular weight between about 400 and 20,000 Daltons, and a load molecule dissociably linked to the poly-cationic moiety wherein the protective chains are linked to polylysine at between 15% and 60% or 35% and 55% of the total amino acid residues on polylysine. In a related embodiment, the invention provides for a pharmaceutical composition.

In another aspect a pharmaceutical composition comprising anyone composition disclosed herein is provided. In a related aspect this invention provides for a method of delivering any load molecule to a subject comprising loading the molecule onto a cationic-core carrier composition comprising a polymeric backbone, a plurality of polymeric protective chains covalently linked and pendant to the polymeric backbone, a plurality of poly-cationic moieties covalently linked and pendant to the polymeric backbone, each with molecular weight of no more than 25% of the average molecular weight of the protective chains, wherein each protective side chain has a molecular weight between about 400 and 20,000 Dalton and administering the composition to said subject.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a schematic representation of one embodiment of the cationic-core composition of the invention: A) protective side chains; B) a polymeric backbone; C) polycationic moieties covalently linked to polymeric core, and D) and anionic load molecule with diameter of 3 nm. The dimension of the carrier is also shown to emphasize that it is greater than the 4 nm glomerular filtration cut off, whereas the carrier and the anionic load molecules together are below this cut off. The following are example of anionic loads and their diameters: siRNA (diameter<3 nm), albumin hydrated (diameter=7.2 nm); growth hormone hydrated (diameter=3 nm); glomerular filtration diameter<4 nm; beta-2 macroglobulin (diameter=3.2 mm); myoglobin (diameter=3.9 nm); hemoglobin (diameter=6.5 nm); gamma globulin (diameter=11.1 nm); and Bence-Jones protein (diameter=5.5 nm).

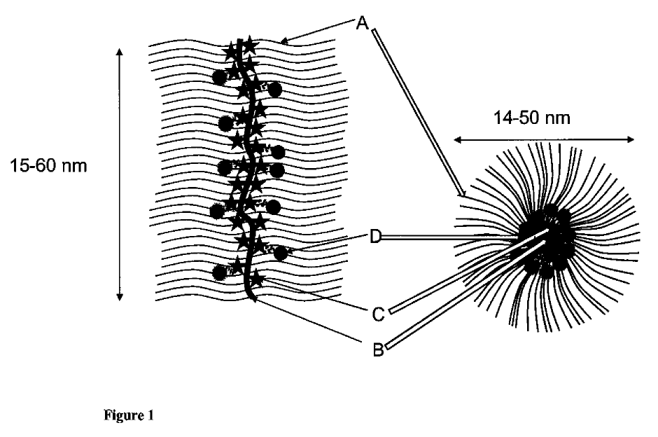

FIG. 2 depicts a diagram of various chemical reactions for making amide bonds that are useful in making the composition of the invention; R₁ can be poly-cationic molecule and R₂ can be polylysine, or polylysine-PEG; or R₁ can be PEG-carboxyl and R₂ can be polylysine, polyethylenimine-polylysine; or R₁ can be polyglutamate or polyaspartate and R₂ can be PEG-amine: a polyamine (such as polyethylenimine; spermine, spermidine, cadaverine, putrescine, polylysine less than 30 mer, polyarginine less than 30 mer); or R₁ can be polyglutamate-PEG or polyaspartate-PEG and R₂ can be a polyamine. EDC is a water soluble version of DCC; both can be used to carry out the reactions.

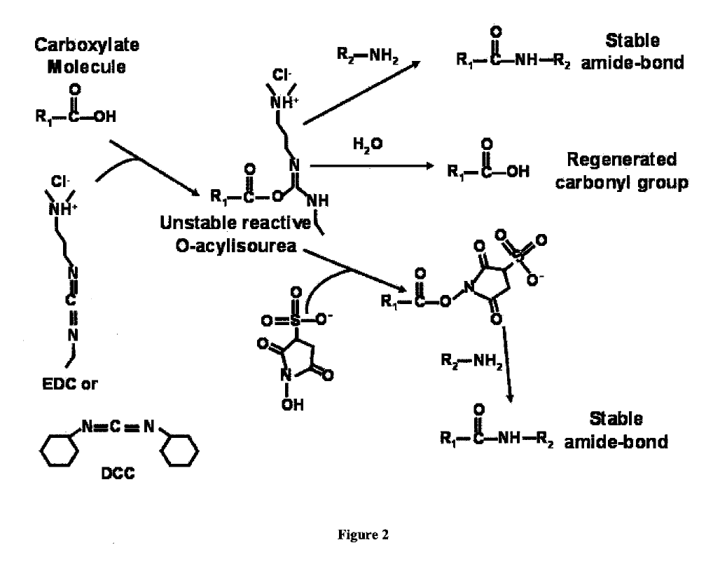

FIG. 3 depicts a diagram of various chemical reactions for attaching poly-cationic amine (R₂) to carrier (R₁) containing functional groups such as isothiocyanate, succinimidyl ester, or sulfonyl chloride. The carrier R₁ can be any backbone polymers. Polymer R₁ can be polyglutamate polyaspartate, polyglutamate-PEG or polyaspartate-PEG.

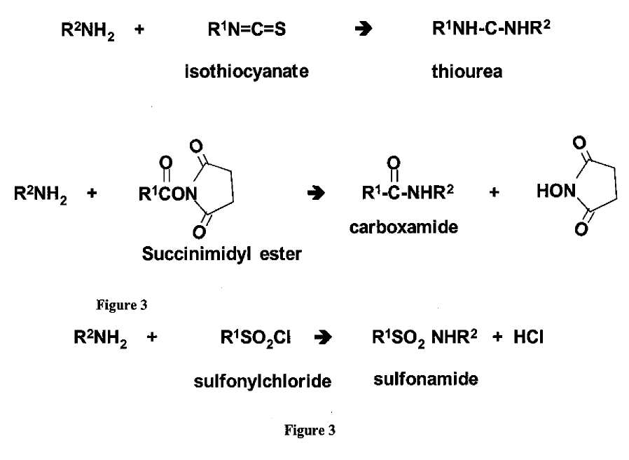

FIG. 4 depicts some of the chemical reactions that may be used to add PEG protective groups, analogs or derivatives thereof, to an amino group containing polymeric backbone.

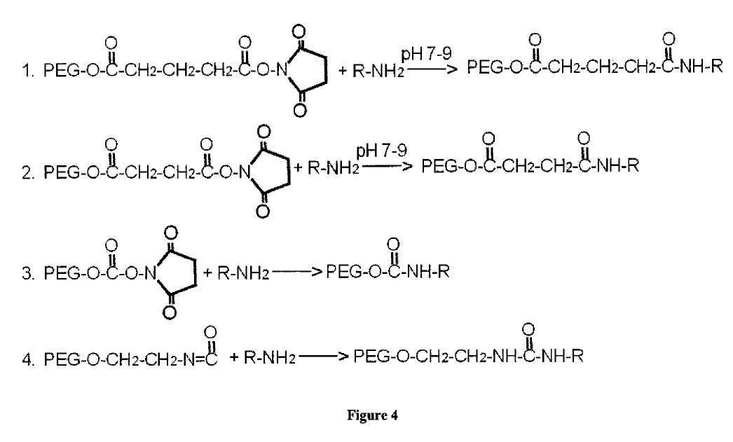

FIG. 5 depicts some of the chemical reactions that may be used to add aldehyde PEG derivatives to an amino group containing polymeric backbone. These are two step condensation-reduction reactions (a & b).

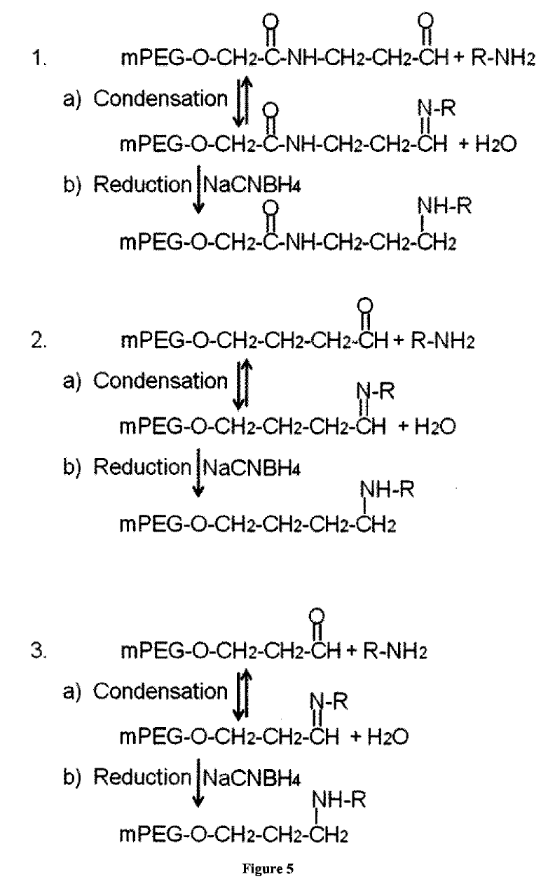

FIG. 6 depicts some of the chemical reactions that may be used to add PEG protective groups, analogs or derivatives thereof, to a hydroxyl containing polymeric backbone.

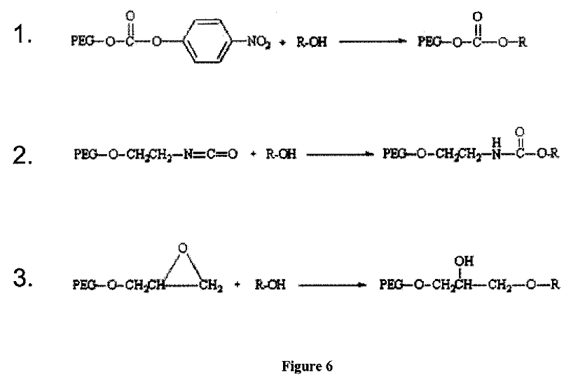

FIG. 7 is the hypothetical free anionic load molecule in the blood with a natural half-life of 20 minutes. There is significant fluctuation in the concentration of anionic load molecule without the carrier. With the carrier, the anionic load molecule will be maintained at therapeutic concentration. The nM concentration of carrier decreases with a half-life of 20 hrs. A) Anionic load molecule level resulting from injection 5 mg/kg, 3 times a day without the carrier of the instant invention, this load molecule has a blood half-life of 20 minutes; B) Carrier along with load molecule has a half-life of 20 hours; C) therapeutic level of free load molecule maintained by the carrier.

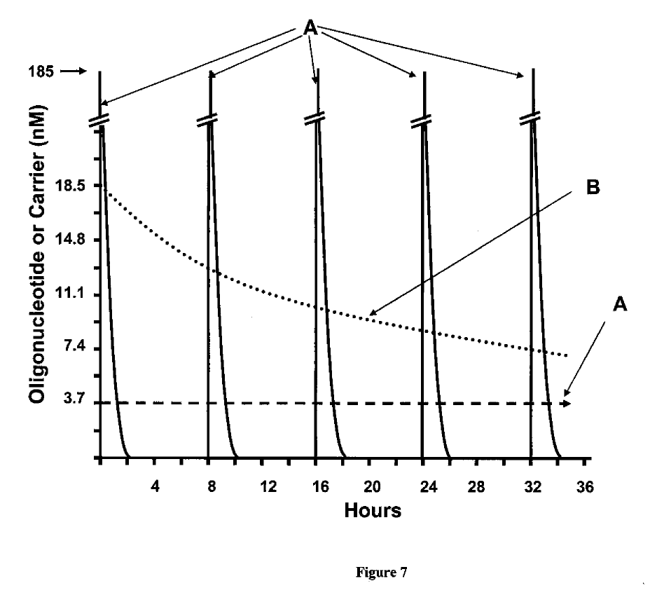

FIG. 8 is a graph showing the theoretical and actual relationship between the amount of amino-group/mg of PLPEG (polylysine-polyethyleneglycol copolymer) and % amino-group saturation of polylysine. This is useful as secondary confirmation of the composition of PLPEG. This PEGylation process is reproducible and adjustable during synthesis by continuing the reaction until the desired % PEGylation is achieved using TNBS amino group assay as a feedback guide during the reaction. The yield is about 50-80% (5-8 gr) of the starting materials. The theoretical prediction was calculated using the following equation: X=[100×(C−Y)]/5YC+C]; where X is the % saturation; Y is the mmol NH₂ per gram of PLPEG as determined by TNBS; C is the mmol of NH₂ per gram of PL (polylysine) as determined by TNBS. The 5 in the term 5YC in the equation represent the size of PEG used which in this case is 5 kDa, thus 5YC. If 10 kDa PEG is used, this will be 10YC. This is useful because once PLPEG product is formed, the percent saturation of the amino group of polylysine can be determined by a single TNBS assay of the final product to determine Y from which X can be calculated.

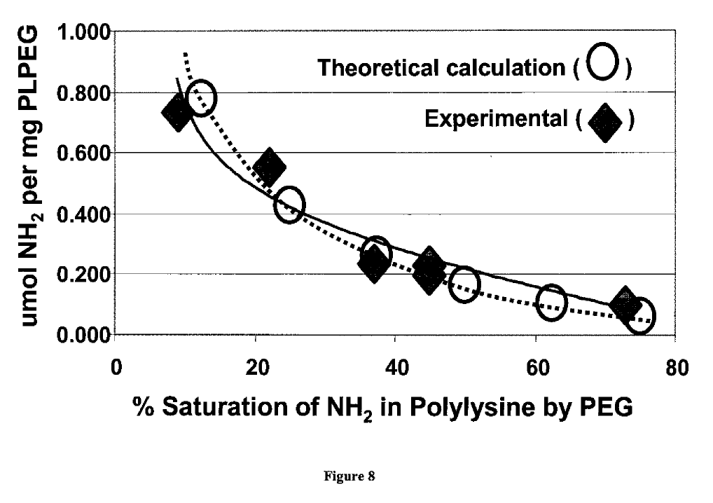

FIG. 9 shows Gel Filtration Chromatograms of the products of the reaction before and after clean up through a 100 kDa MWCO membrane (Amersham Biosciences, Needham, Mass.) showing that all unreacted PEG had been removed. The column used was Ultrahydrogel linear (0.78×30 cm, Waters) eluted at flow rate of 0.6 ml/min PBS. The materials were detected using a refractive index detector. Panel A is 20PLPEG5-55 (20 kDa polylysine where 55% of the amino groups were reacted with PEG succinate of 5 kDa molecular weight) prior to clean-up from unreacted 5 kDa PEG. Panel B is 5 kDa PEG alone. Panel C is 20PLPEG5-55 after clean up.

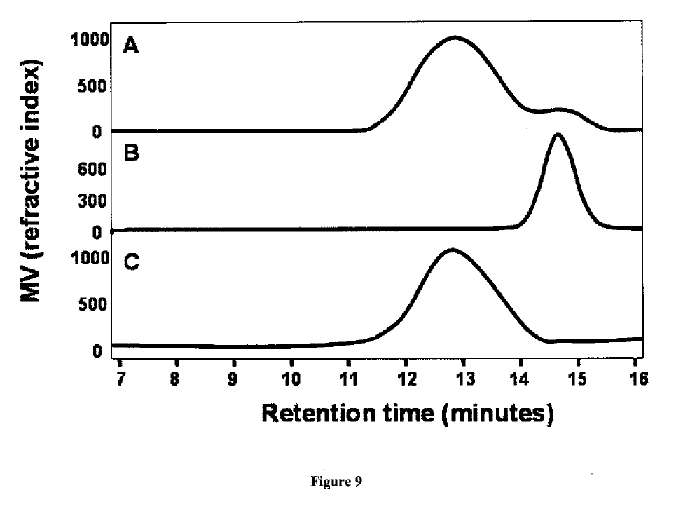

FIG. 10 shows the Stokes radii of various carriers along with proteins of known Stokes radii. These were analyzed on the Ultrahydrogel Linear column (0.78 cm diameter×30 cm length) using PBS with 15% Acetonitrile at a flow rate of 0.6 nm/min as mobile phase. The 20PL-PEG5-55 (20 kDa polylysine where 55% of the amino groups were reacted with PEG succinate of 5 kDa molecular weight), 40PL-PEG5-30 (40 kDa polylysine where 30% of the amino groups were reacted with PEG succinate of 5 kDa molecular weight), 40PL-PEG5-51 (40 kDa polylysine where 51% of the amino groups were reacted with PEG succinate of 5 kDa molecular weight), and 40PL-PEG5-27 (40 kDa polylysine where 27% of the amino groups were reacted with PEG succinate of 5 kDa molecular weight) are larger than the glomerular filtration cut off that is above 4 nm (40 Angstrom) in diameter (or 20 Angstrom in radius). Proteins with known Stokes radii were used as reference including Thyroglobulin (669 kDa; 85.5 Angstroms stokes radius), Catalase (248 kDa; 52.2 Angstrom Stokes radius), and BSA (67 kDa; 35.5 Angstroms stokes radius), Catalase (248 kDa; 52.2 Angstrom Stokes radius), and BSA (67 kDa; 35.5 Angstrom Stokes radius).

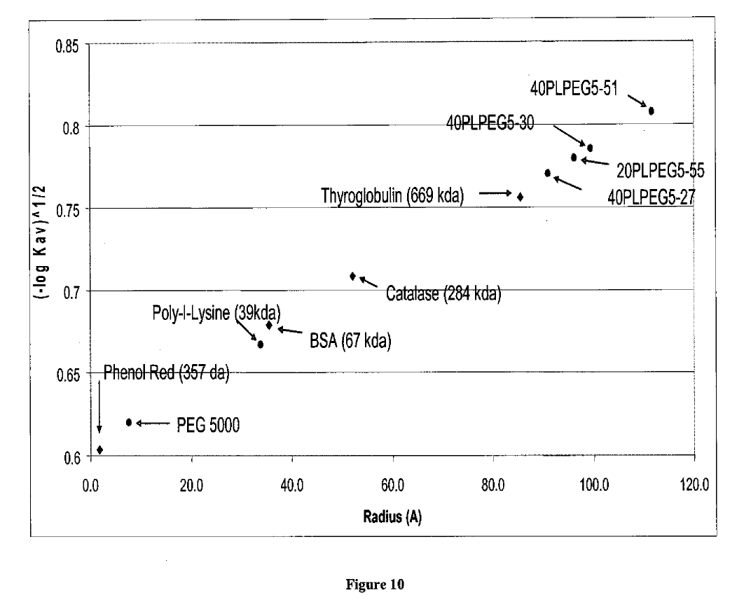

FIG. 11 shows gel permeation chromatograms of 50 ul of (A) 10 mg/ml carrier comprising polylysine (40+/−20 kDa Mw), methoxypolyethylene glycol or MPEG (5 kDa) covalently linked and mostly pendant to polylysine, and branched polyethyleneimine or PEI (400 Da) covalently linked and mostly pendant to polylysine, (B) 0.8 mg/ml double stranded 23 nm er DNA polynucleotide, and (C) mixture of carrier (10 mg/ml) in A and double stranded 23mer DNA (0.8 mg/ml) in B. The polynucleotide has greater absorbance at 260 nm than the carrier. The carrier has 40% of polylysine amino group covalently linked to MPEG and the remaining amino groups are linked to the branched polyethyleneimine with molecular weight average of 400+/−100 Da though a succinate spacer. The carrier has an average diameter of 20 nm, and comes out at the void volume (Vo=5.7 ml or 5.7 min) of BioSep2000SEC (Phenomenex) when eluted with PBS, indicating its large size. The 23 mer polynucleotide comes out at the included volume (7.5 minutes) with some aggregates coming out at the void volume. When combined, all the 23 mer polynucleotide comes out at the void volume indicating its large size. The size however is not larger than 50 on (not shown) as determined using Tosoh G4000WXL column (78×30 cm; with PBS/15% Acetonitrile mobile phase flowing at 0.6 ml/min) indicating that in the presence of oligonucleotide there is no formation of supramolecular structure. This is a clear indication that the carrier binds to polynucleotide with high affinity. Unlike the mixture of polynucleotide and PEI which precipitate and becomes cloudy, the solution injected was completely soluble and no cloudiness was observed confirming that there is no carrier aggregation (or formation of supramolecular structure; i.e. structure with diameter greater than 70 nm or more commonly 100-200 nm in diameter) as a result of loading the 23-mer polynucleotide.

FIG. 12 shows that the binding of a poly-cationic core carrier to polynucleotide is sequence independent. Shown is the result of binding experiment showing that random DNA fragments (8 ug/ml) loaded into 0.4 mg/ml carrier 40PLPEG540-PEI-12 (see example 5) in PBS. The total DNA loaded is represented by the first Bar and the total DNA bound is the total DNA loaded minus the free shown in the second bar. The free was determined from the filtrate after filtration of 250 ul sample using 100 kDa molecular weight cut off filter (Microcon Ultracell YM-100, made up of regenerated cellulose from Millipore, Bedford, Mass.). The mixture containing the carrier and random DNA fragment was filtered by centrifugation at 12,000×g for 12 minutes and the filtrate was assayed for DNA using DNA Assay kit (Quant-iT ds DNA from Invitrogen, Eugene, Oreg.) according to manufacturer's instruction. The sample that went through the filter is the amount of free or unbound DNA as the carrier is too large to penetrate the filter. The dsDNA in the filtrate binds fluorescent dye in the kit that fluoresces (excitation 485 nm/emission 535 nm) at intensity proportional to the amount of DNA in the sample. Unfiltered sample without carrier was assayed to represent the total DNA loaded and the result is similar to the filtrate of the sample without carrier indicating that the filter does not retain DNA. This results shows that the carrier binds DNA very strongly. Because the DNA used is a random mixture of many different sequences, the results indicate that the binding is sequence independent and can be used for any polynucleotide independent of sequence.

FIG. 13 is a graph that shows the impact of 6.25 nM siRNA against EGR-1 mRNA on the EGR-1 mRNA level after 4 hours in the presence or absence of RNA carrier (40PLPEG540PEI4; lot#20071005). The levels of EGR-1 mRNA in INS cells are shown as multiple of untreated control value of 1 in the Y-axis. For complete procedure and explanation about the experiment to obtain this graph, see example 20 below.

FIG. 14 shows the decrease in insulin concentration in INS cell culture supernatant after treatment with siRNA against EGR1 gene. Cells treated with siRNA against EGR1 gene and those not treated with siRNA (diluents control) has the same viability as determined by trypan blue exclusion. Using t-test the free siRNA without a carrier did not significantly decrease insulin at 44 hour time point whereas the carrier (40PLPEG540PEI4; lot#20071005) formulated siRNA significantly decrease insulin at 44 hour time point (p<0.05). At 20 and 28 hours after treatment with a carrier formulated with siRNA, a significant decrease in insulin was observed (P<0.01) compared with diluents control and unformulated siRNA.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### DEFINITIONS

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

The articles “a” and “an” are used to refer to one or to more than one (i.e., to at least one) of the grammatical object of the article. By way of example, “an element” means one element or more than one element.

The term “carrier” of the present invention means a composition capable of dissociably binding a load molecule or a therapeutic agent with high affinity (dissociation constant or Kd of 10 micromolar or less) so as to slowly provide a free load molecule or a free therapeutic agent by slowly dissociating from the composition.

The term “derivative” or “analog” as used herein refers to a compound whose core structure is the same as, or closely resembles that of, a parent compound, but which has a chemical or physical modification, such as a different or additional groups. The term also includes a peptide with at least 50% sequence identity with the parent peptide. The term also includes a peptide with additional groups attached to it compared to the parent peptide, such as oligonucleotides and/or additional amino acids that do not exceed the mass of the original parent peptide. The term also includes a polymer with additional group attached to it, such as alkoxy group, compared to the parent polymer. The term also includes methoxylated oligonucleotides with additional methoxy group(s) attached to it compared to the parent oligonucleotide chain. The term also includes hydroxylated oligonucleotides with additional hydroxy group(s) attached to it compared to the parent oligonucleotide chains.

The term DNA also includes fragment of this materials such as antisense DNA oligonucleotides. For the purpose of the present invention small segments of DNA or RNA (less than about 200 bases) can also be referred to as an oligonucleotide. The term “locked nucleic acid” or LNA, refers to a modified RNA with a nucleotide that is inaccessible. The ribose moiety of a LNA nucleotide is modified with an extra bridge connecting the 2′ and 4′ carbons. The bridge “locks” the ribose in 3′-endo structural conformation, which is often found in A-form of DNA or RNA. LNA nucleotides can be mixed with DNA or RNA bases in the oligonucleotide whenever desired. Such oligomers are commercially available. The locked ribose conformation enhances base stacking and backbone pre-organization. This increases significantly the thermal stability (melting temperature) of oligonucleotides.

The term “MicroRNA” or “miRNA” are a related class of gene regulatory small RNAs, typically 21-23 nt in length. They typically differ from siRNA because they are processed from single stranded RNA precursors and show only partial complementarity to mRNA targets. They have been implicated in a wide range of functions such as cell growth and apoptosis, development, neuronal plasticity and remodeling, and even insulin secretion. miRNAs have also been implicated in disease: e.g. an overabundance of miRNA has been reported in cases of Fragile X Mental Retardation, while some cancers are associated with up- and downregulation of certain miRNA genes. Initial studies have indicated that miRNAs regulate gene expression post-transcriptionally at the level of translational inhibition at P-bodies in the cytoplasm. However, miRNAs may also guide mRNA cleavage similar to siRNAs. This is often the case in plants where the target sites are typically highly complementary to the miRNA. While target sites in plant mRNAs can be found in the 5′UTR, open-reading frames and 3′UTR, in animals it is the 3′ UTR that is the main target. This difference between plants and animals may be explained by their different modes of gene silencing. miRNAs are first transcribed as part of a primary microRNA (pri-miRNA). This is then processed by the Drosha with the help of Pasha/DGCR8 (=Microprocessor complex) into pre-miRNAs. The 75 nt pre-miRNA is then exported to the cytoplasm by exportin-5, where it is then diced into 21-23 nt siRNA-like molecules by Dicer. In some cases, multiple miRNAs can be found on the pri-miRNA. Some of these miRNA can be used as therapeutic agent and it is understood that the present invention includes these materials as load molecules.

The term “oligonucleotide” means short sequences of polynucleotides such as oligoribonucleotides (ribonucleic acid (RNA) or oligodeoxyribonucleotides (deoxyribonucleic acid (DNA), typically with twenty or fewer bases (or nucleotide units) but can be up to up to 160 to 200 bases. Each nucleotide monomer is made up of 3 components covalently linked to each other: a nitrogenous base (purine or pyrimidine), a five-carbon sugar and, a phosphate group. Frequently the term nucleotide unit is also called base because each nucleotide unit has one nitrogenous base. Oligonucleotides can easily be made synthetically and the length of a synthesized oligomer is usually denoted by ‘mer’ (from ‘Greek’ meros “part”). For example, a fragment of 25 bases would be called a 25-mer. Oligonucleotides are often used as probes for detecting complementary DNA or RNA because they bind readily to their complements. Oligonucleotides have never been used for the purpose of loading the poly-cationic core carrier of the present invention. Oligonucleotides are sometimes referred to as oligos. Antisense oligonucleotides are single strands of DNA or RNA that are complementary to a chosen sequence. In the case of antisense RNA they prevent translation of complementary RNA strands by binding to it. Antisense DNA can be used to target a specific, complementary (coding or non-coding) RNA. If binding takes places this DNA/RNA hybrid can be degraded by the enzyme RNase H. Synthesis of oligonucleotide is well known to those skilled in the art and some of these methods are outlined below. Oligonucleotide may comprise of several nucleotides linked together where each nucleotide can be anyone of 2′-deoxyribonucleotide, ribonucleotide, 2′-O-methylribonucleotide, locked ribonucleotide, N-(2-ethylamino)glycine nucleotide and morpholino nucleotide. Each nucleotide can be linked to another by 3′-5′ or 2′-5′ linkage, wherein the linkage can be phosphodiester, phosphorothio, phosphotriester, phosphorodiamidate and a peptide. The base in each nucleotide of oligonucleotide can be anyone of the typical bases found in nucleic acid such as adenine, thymidine, guanine, cytosine, and uracil or it can also be anyone of the atypical bases such as inosine, thioinosine, thiouridine, xanthosine, pseudouridine, or orotidine.

The term “polynucleotide” as used herein refers to an organic polymer molecule comprised of nucleotide monomers covalently bonded in a chain. DNA (deoxyribonucleic acid), RNA (ribonucleic acid), and their analogs or derivatives are examples of polynucleotides. Polynucleotide also includes a polymer containing both types of nucleotides (deoxyribonucleotide and ribonucleotide). Oligonucleotide is also a polynucleotide but not all polynucleotides are oligonucleotides. Locked nucleic acid, siRNA, and miRNA are also polynucleotides.

The term “Small interfering RNA” or “siRNA”, sometimes known as short interfering RNA or silencing RNA, are a class of 20-25 nucleotide-long double-stranded RNA molecules that play a variety of roles in biology. siRNA is involved in the RNA interference (RNAi) pathway where the siRNA interferes with the expression of a specific gene. In addition to their role in the RNAi pathway, siRNAs also act in RNAi-related pathways, e.g. as an antiviral mechanism or in shaping the chromatin structure of a genome; the complexity of these pathways is only now being elucidated. SiRNAs have a well defined structure: a short (usually 21-nucleotides) double-strand of RNA (dsRNA) with 2-nucleotide 3′ overhangs on either end: Each strand has a 5′ phosphate group and a 3′ hydroxyl (—OH) group. This structure is the result of processing by dicer, an enzyme that converts either long dsRNAs or small hairpin RNAs into siRNAs (Bernstein et. al. 2001 Nature 409 (6818): 363-6). SiRNAs can also be exogenously (artificially) introduced into cells by various transfection methods to bring about the specific knockdown of a gene of interest. Essentially any gene of which the sequence is known can thus be targeted based on sequence complementarity with an appropriately tailored siRNA. This has made siRNAs a tool for gene function and drug target validation studies in the post-genomic era. Transfection of an exogenous siRNA can be problematic, since the gene knockdown effect is only transient, particularly in rapidly dividing cells. The present invention is designed to overcome this and satisfy the long felt need to provide a sustained inhibition that remains non-permanent.

Polymeric Backbone

The polymeric backbone of the present disclosure provides multiple sites from where the poly-cationic chains and hydrophilic protective chains can be attached. In some embodiments the polymeric backbone is a homo- or heteropolymer. In certain embodiments the polymeric backbone is a straight chain homopolymer. In other embodiments the polymeric backbone is a branched polymer. In specific embodiments the polymeric backbone is a polyaminoacid, a polymer with repeating amino acids. The amino acids may be of natural or synthetic origin. In one embodiment the polymer is non-proteinaceous, that is either a polyaminoacid that is not naturally made by a living organism unless recombinantly engineered or a polyamino acid that does not have activity associated with its three dimensional conformation. Non-limiting examples of non-proteinaceous polyamino acids are poly-(L and/or D)-lysine, poly-(L and/or D)-glutamate, poly-(L and/or D)-glutamate, poly-(L and/or D)-aspartate, poly-(L and/or D)-serine, poly-(L and/or D)-threonine, poly-(L and/or D)-tyrosine, and poly-(L and/or D)-arginine. The non-proteinaceous polyamino acids also includes polyamino acids with R-groups that are not naturally occurring but contains carboxyl, amino, hydroxyl, or thiol groups that can provide repeating functional groups that are modifiable for the attachment of protective groups, oligonucleotides and poly-cationic moieties. The non-proteinaceous polyaminoacids are among the possible backbone component of the invention.

In certain embodiments the polymeric backbone is a polyamino acid which may have D- or L-chirality or both. In specific embodiments, the polymeric backbone is a straight chain polyaminoacid such as but not limited to polylysine, polyornithine, polyarginine, polyglutamate, polyaspartate, polyserine, polythreonine, polytyrosine or any other amide linked heteropolymer made from amino acids. In other embodiments, another polymeric backbone with repeating modifiable functional groups may also be used such as but not limited to those with repeating sulfhydryl (thiol), phosphate, and hydroxyl groups. Carbohydrate polymers and other synthetic polymers where monomers are non-biological may also be used as the polymeric backbone.

The polymeric backbone may have a molecular weight of 500-1,000,000 Daltons, or 500-10,000 Daltons, 5-100,000 Daltons, or 1000-10,000 Daltons, or 10,000-50,000 Daltons, or 10,000-100,000 Daltons, or at least 1000 Daltons, or at least 5000 Daltons, or at least 10,000 Daltons, or at least 25,000 Daltons, or at least 50,000 Daltons, or at least 75,000 Daltons, or at least 100,000 Daltons, or at least 250,000 or at least, 500,000 Daltons, or at least 750,000 or at least, or at most 1000 Daltons, or at most 5000 Daltons, or at most 10,000 Daltons, or at most 25,000 Daltons, or at most 50,000 Daltons, or at most 75,000 Daltons, or at most 100,000 Daltons, or at most 250,000 or at most, 500,000 Daltons, or at most 750,000 or at most 1,000,000 Daltons.

The % of derivitization (the % of residues on the polymeric backbone substituted with for example protective chains or poly-cationic moieties) of a polymeric backbone can vary from 15%-70%. In certain embodiments the backbone is substituted 15%-70%, or 15%-60%, or 15%-40%, or 35%-55%, or at least or at most 20%, or at least or at most 25%, or at least or at most 30%, or at least or at most 35%, or at least or at most 40%, or at least or at most 45%, or at least or at most 50%, or at least or at most 55%, or at least or at most 60%, or at least or at most 65%, or at least or at most 70% of the total residues on the polymer. In specific embodiments the degree of derivitization of a polyaminoacid backbone with protective chains or poly-cationic moieties is 15%-70%, or 15%-60%, or 15%-40%, or 35%-55%, or at least 20%, or at least 25%, or at least or at most 30%, or at least or at most 35%, or at least or at most 40%, or at least or at most 45%, or at least or at most 50%, or at least or at most 55%, or at least or at most 60%, or at least or at most 65%, or at least or at most 70% of the total amino acid residues on the polyaminoacid.

By way of example, possible backbones can include but are not limited to polylysine, polyaspartic acid, polyglutamic acid, polyserine, polythreonine, polycysteine, polyglycerol, natural saccharides, aminated polysaccharides, aminated oligosaccharides, polyamidoamine, polyacrylic acids, polyalcohols, sulfonated polysaccharides, sulfonated oligosaccharides, carboxylated polysaccharides, carboxylated oligosaccharides, aminocarboxylated polysaccharides, aminocarboxylated oligosaccharides, carboxymethylated polysaccharides, and carboxymethylated oligosaccharides.

Poly-Cationic Moiety

Poly-cationic moieties of the present disclosure provide positively charged groups to the polymeric backbone. In certain embodiments, the poly-cationic moiety is pendant to the polymeric backbone. The term pendant as used herein refers to one or more moieties branching from a linear polymeric chain or a linear portion of a branched polymer. In the context of the present invention, it is the polycationic group attached or branching from the polymeric backbone.

In other embodiments, the poly-cationic moiety is attached to the terminal of a polymeric backbone. Polycationic moieties are generally positively charged moieties, often nitrogen-containing or amine-containing moieties. In one embodiments the poly-cationic moiety is an amine-containing moiety. In another embodiment it is an amine-containing linear alkyl or substituted alkyl moiety. An exemplary poly-cationic moiety is polyethyleneimine (PEI). Other exemplary poly-cationic moieties include but are not limited to, polylysine, polyornithine, polyarginine, polyhistidine, polyhistidine at ph<7, spermine, spermidine, or any other polyamine that will provide positively charged amino groups pendant to the polymeric backbone.

In certain embodiments the poly-cationic moiety pendant to the polymeric backbone has molecular weight between 40-5000 Daltons, 40-4000 Daltons, 40-3000 Daltons, 40-2000 Daltons, or 40-1500 Daltons, or 40-500 Daltons, or 100-2000 Daltons, or 100-500 Daltons or at least 90 Daltons, or at least 1250 Daltons, or at least 25000 Daltons, or at least 3000 Daltons, or 5000 Daltons. In specific embodiments the poly-cationic moiety is PEI, is pendant to the polymeric backbone, and has molecular weight of between 40-5000 Daltons, or 40-4000 Daltons, or 40-3000 Daltons, or 40-2000 Daltons, or 40-1500 Daltons, or 40-1000 Daltons, or 100-3000 Daltons, or 100-2000 Daltons, or 100-1000 Daltons, or 100-500 Daltons or at least 90 Daltons, or at least 400 Daltons, or at least 800 Daltons, or at least 1200 Daltons, or at least 2000 Daltons, or at least 2500 Daltons, or at least 3000 Daltons, or 5000 Daltons. In other embodiments the poly-cationic moiety will provide at least 2, or 2-200, or 2-150, or 2-100, or 2-75, or 2-50, or 2-40, or 2-30, or 2-20, or 2-15, or 2-10, or 2-5, or 3-50, or 340, or 3-30, or 3-20, or 3-15, or 3-10, or 3-5, or least 2, or at least 3, or at least 5, or at least 10, or at least 15, or at least 20, or at least 30, or at least 40, or 50 positively charged amino groups pendant to the polymeric backbone.

Protective Hydrophilic Groups/Protective Chains

As used herein, a protective side chain refers to a molecule(s) which protects a carrier molecule and an anionic load molecule from contact with other macromolecules due to extensive linking or binding of hydrophilic molecules, such as but not limited to water. Because of this extensive binding with water molecules, the protective chain also increases water solubility of the composition. The protective chains do not have significant amounts of charge but are water soluble making the poly-cationic core composition non-immunogenic. This also means that protective chain provides a hydrophilic property to the composition. The term “protective side chain” is used interchangeably with the terms “protective group” and “protective chain”.

In certain embodiments a protective chain or protective hydrophilic group is useful because it: 1) facilitates the solubility the composition even while maintaining a high drug payload; 2) assists in the formation of a stearic barrier which can prevent load molecules (oligonucleotide, RNA, DNA, anionic peptides, anionic proteins and other anionic drugs and therapeutic agents) from binding or interacting with other macromolecules, enzymes (nucleases and proteases) and cells in the environment; 3) provides load molecules (oligonucleotide, RNA, DNA, anionic peptides, anionic proteins other anionic drugs and therapeutic agents) with longer circulation times or biological half-lives in vivo (e.g. for decreasing glomerular filtration in kidneys, decreasing kidney and liver uptake, decreasing macrophage uptake, etc.) and creates a circulating depot; 4) decreases undesirable immunogenicity of the carrier or its load molecules such as a oligonucleotide, RNA, DNA, anionic peptides and anionic proteins and other anionic drugs and therapeutic agents; and/or 5) increases the size of the carrier to take advantage of the abnormal permeability of tumor vessels and faceplates the accumulation of the carrier with load molecules in a tumor or inflammation site by delivering the load molecules or anti-tumor compounds to the tumor which is especially useful for treating tumors.

In certain embodiments, the protective chains of the present disclosure are large enough to protect the poly-cationic moiety from exposure to the environment. In specific embodiments where the size of each protective chain in the compound is roughly the same, then each polycationic moiety has a mass of no more than about 25% of the mass of a protective side chain. In other embodiments, each polycationic moiety has a mass of no more than about 25% of the average mass of the protective side chains in a composition.

Exemplary protective chains of the poly-cationic core carrier composition of the present disclosure include but are not limited to: polyethylene glycol (PEG, a polymer of ethylene oxide), polypropylene glycol, methoxypolyethylene glycol, methoxypolypropylene glycol, a co-polymer of polyethylene glycol and polypropylene glycol; or an alkoxy derivative thereof. In other embodiments, the protective chain is one of methoxypolyethylene glycol, methoxypolypropylene glycol, or a co-polymer of methoxypolyethylene glycol and methoxypolypropyleneglycol. The protective chain may also be polyethylene glycol monoamine, methoxypolyethylene glycol monoamine, methoxy polyethylene glycol hydrazine, methoxy polyethylene glycol imidazolide or a polyethylene glycol diacid. In certain embodiments methoxylated or ethoxylated polysaccharides can also be used as protective as alkoxylation can reduce or eliminate immunogenicity.

A protective chain or chains are linked to the polymeric backbone or poly-cationic pendant to the polymeric backbone preferably by a single linkage. The term pendant as used herein refers to one or more moieties branching from a linear polymeric chain or a linear portion of a branched polymer. In the context of the present invention, it is the protective group attached or branching from the polymeric backbone.

Protective side chains of the present disclosure provide protection to the polymeric backbone and/or the poly-cationic moieties. In certain embodiments, the protective side chain is pendant to the polymeric backbone. In other embodiments, the protective side chain is attached to the terminal of a polymeric backbone. In yet other embodiments the protective side chain is pendant to the polycationic moieties. In specific embodiments the protective chain or chains are linked to the polymeric backbone or poly-cationic moiety by a single linkage. In some embodiments, a second protective group is covalently linked to a poly-cationic moiety. In some embodiments only one protective group is used to protect the poly-cationic core composition. In other embodiments, two, three, or more different protective groups are used to protect the poly-cationic core composition.

In certain embodiments of the present disclosure, the protective chain is non-ionic. In certain other embodiments of the present disclosure each protective chain has a molecular weight of about 2000-20,000 Daltons, or 5,000-10,000 Daltons, or 10,000-20,000 Daltons, or at least 2000 Daltons, or at least 5000 Daltons, or at least 10,000 Daltons, or at least 12,000 Daltons, or at least 15,000 Daltons, or at most 2000 Daltons, or at most 5000 Daltons, or at most 10,000 Daltons, or at most 12,000 Daltons, or at most 15,000 Daltons or at most 20,000 Daltons.

Anionic Load Molecules

The term load molecule as used herein encompasses any molecule with high affinity (those with affinity constant (Ka) of greater than 0.1 million/molar or dissociation constant (Kd) of less than 10 micromolar) to the carrier, allowing it to be loaded into the carrier. The affinity constant or dissociation constant can easily be ascertained by those skilled in the art and examples are provided in this disclosure. For the purpose of the present invention, these load molecules include but are not limited to anionic peptides (50 or less amino acids), anionic proteins (greater than 50 amino acids), polynucleotide (RNA, DNA or their analogs). In certain embodiments, anionic peptides and proteins have isoelectric points of less than pH of 7. Other peptides and proteins that are not highly anionic can be made anionic by attaching a degradable oligonucleotide such as RNA and the process of such a modification is very well known in the art.

In certain embodiments the load molecule is a therapeutic agent, any chemical moiety that is a biologically, physiologically, or pharmacologically active and act locally or systemically in a subject. For the purpose of the present invention, a therapeutic agent loaded into the carrier is can have anionic groups such as oligonucleotides or have oligonucleotide group modification, if not otherwise anionic. In one embodiment, anionic load molecules can be polynucleotides such as ribonucleic acid (RNA), deoxyribonucleic acid (DNA), oligoribonucleotide, oligodeoxyribonucleotide, small interfering RNA (siRNA), microRNA, and derivatives thereof. In other embodiments, aniono load molecules can be anionic peptides, anionic proteins, anionic carbohydrates; anionic lipids, and anionic proteoglycans. In yet other embodiments the anionic load molecule can be an anionic group containing imaging agent, an anionic group containing therapeutic agent, anionic peptide, an anionic protein such as a cytokine, lymphokine, hormone, hormone agonist, hormone antagonist, antibiotic, analgesic, toxin, photo-toxin, cytostatic agent, cytotoxic agent, psychotropic agent, steroidal anti-inflammatory agent, non-steroidal anti-inflammatory agent, immunosuppressive agent, anti-bacterial agent, anti-viral drug, anti-fungal drug, chelator, vitamin, protease inhibitor, pesticide, aminoglycoside, polymyxin, ACE inhibitor, peptide, protein, antibody, antibody fragment, recombinant peptide, peptide isolated from plants, peptide isolated from fingi, peptide isolated from animals, peptide isolated from bacteria, peptide isolated from viruses, peptides isolated from cells in culture, synthetic peptide, peptidomimetic compound, organic compound, synthetic organic compound, organic compound isolated from plants, organic compound isolated from fungi, organic compound isolated from animals, organic compound isolated from bacteria, organic compound isolated from viruses, organic compound isolated from cells in culture, and organometallic compound. The load molecules of the present invention also include therapeutic agents derivatized to contain anionic groups or a naturally anionic therapeutic agent. These includes siRNA, antisense-DNA, antisense-RNA, glucagon-like-peptide, glucagon-like-peptide derivatives, exenatide, glucagon-like-peptide-1, glucagon-like-peptide-2, leptin, leptin fragment, Gastric inhibitory polypeptide(GIP), Epidermal Growth Factor (EGF) receptor ligand, EGF, Transforming Growth Factor alpha (TGF-alpha), Betacellulin, Gastrin/Cholecystokinin receptor ligand, Gastrin, Cholecystokinin, lysostaphin, interferon, interferon gamma, interferon beta, interferon alpha, interleukin-1, interleukin-2, interleukin-4, interleukin-6, interleukin-8, interleukin-10, interleukin-12, tumor necrosis factor, tumor necrosis factor alpha, tumor necrosis factor beta, auristatin, nisin, insulin, insulin-like growth factor, growth hormone, growth hormone releasing hormone (GHRH), nerve growth factor, brain-derived neurotrophic factor, enzymes, endostatin, angiostatin, thrombospondin, urokinase, streptokinase, blood clotting factor VII, blood clotting factor VIII, granulocyte-macrophage colony-stimulating factor (GM-CSF), granulocyte colony-stimulating factor (G-CSF), thrombopoietin, calcitonin, parathyroid hormone (PTH) and its fragments, erythropoietin, atrial natriuretic factor, monoclonal antibodies, monoclonal antibody fragments, somatostatin, protease inhibitors, adrenocorticotropin, gonadotropin releasing hormone, oxytocin, leutinizing-hormone-releasing-hormone, follicle stimulating hormone, glucocerebrosidase, thrombopoietin, filgrastin, prostaglandins, epoprostenol, prostacyclin, cyclosporine, vasopressin, terlipressin, desmopressin, cromolyn sodium (sodium or disodium chromoglycate), vasoactive intestinal peptide (VIP), vancomycin, antimicrobials, polymyxin b, anti-fungal agents, anti-viral agents, enfuvirtide, doxorubicin, etoposide, fentanyl, ketamine, and vitamins.

Any anionic molecules may be attached to therapeutic agents to facilitate loading or improve affinity to the carrier. The anionic groups attached to the therapeutic agent can be RNA, DNA, or any molecule containing at least two of sulfate, phosphate, or carboxyl groups in close proximity (about 2-10 chemical bonds apart) to each other or their analogs. In certain embodiments, RNA will be attached as the anionic group as once released from protective carrier, RNA will rapidly be removed by endogenous RNAses leaving the native unaltered therapeutic agent.

An exemplary anionic group to attach to therapeutic peptides and proteins to increase their negative charge are ribonucleotides as they are less stable than deoxyribonucleotides. Use of RNA can facilitate loading into the carrier during the formulation and once released into the blood, nucleases will degrade the attached RNA. In certain embodiments, depending on the intended purpose, the higher stability of the attached nucleotide may be desired and deoxyribonucleotide may be used in those circumstances.

In certain embodiments, the load molecule is an aptamer, oligonucleic acids or peptide molecules that bind a specific target molecule through specific folding. One of the embodiments of the present invention is to deliver nucleic acid aptamers by providing the carrier with poly-cationic groups that can bind the polyphosphate regions of a nucleic acid aptamer. In certain embodiments aptamers are created by selecting them from a random sequence pool. In other embodiments the aptamers are natural, such as riboswitches. In specific embodiments, aptamers can be combined with ribozymes to self-cleave in the presence of their target molecule. In certain embodiments RNA and DNA aptamers are nucleic acid species that have been engineered for example through repeated rounds of in vitro selection or equivalently, SELEX (systematic evolution of ligands by exponential enrichment) to bind to various molecular targets such as small molecules, proteins, nucleic acids, and even cells, tissues and organisms. Aptamers offer the utility for biotechnological and therapeutic applications as they offer molecular recognition properties that are comparable to that of antibodies. This is possible through specific folding to create recognition sites. Although this folding can be interrupted by binding to the carrier, upon release from the carrier re-folding will occur to provide aptamers that has the right folding to be biologically or therapeutically active. In addition to their discriminate recognition, aptamers offer advantages over antibodies as they can be engineered in vitro, are produced by chemical synthesis, possess desirable storage properties, and elicit little or no immunogenicity in therapeutic applications. There is no systematic difference between RNA and DNA aptamers, except for the greater intrinsic chemical stability of DNA. Non-modified aptamers are cleared rapidly from the bloodstream, with a half-life of minutes to hours, mainly due to nuclease degradation and clearance from the body by the kidneys, a result of the aptamer's inherently low molecular weight. Several modifications, such as 2′-fluorine-substituted pyridines, polyethylene glycol (PEG) linkage, etc. (both of which are used in Macugen, an FDA-approved aptamer) are available to scientists with which to increase the half-life of aptamers easily to the day or even week time scale. Certain embodiments of the present invention deliver unmodified nucleic acid aptamers by providing a carrier with poly-cationic moieties. In other embodiments a peptide aptamer is designed to interfere with other protein interactions inside cells. In exemplary forms, they can consist of a variable peptide loop attached at both ends to a protein scaffold. This double structural constraint greatly increases the binding affinity of the peptide aptamer to levels comparable to an antibody's (nanomolar range). The variable loop length is typically comprised of about 5 to 25 amino acids, and the scaffold may be any protein which has good solubility (which for the purpose of the present disclosure will preferably anionic) and compact properties. These peptide aptamers can be made to contain oligonucleotide, such as RNA for rapid removal in vivo, to be able to load into compositions provided by this disclosure by phosphate-poly-cation interaction. In certain embodiments the aptamer is loaded into the carrier and it is protected from degradation due to high density of protective chain shielding.

Hydrophobic Groups

In certain embodiments of the present disclosure, the poly-cationic core composition is further linked to hydrophobic functional groups. In specific embodiments the hydrophobic groups are covalently attached to the poly-cationic moiety. A hydrophobic group refers to a molecule or several molecules, chemical moieties or portion of a molecule which is non-polar and provides a hydrophobic environment for a load molecule to interact in order to avoid the surrounding aqueous environment. Hydrophobic groups may be aliphatic hydrocarbon chains and/or ring compounds that do not have positive or negative charge and are capable of binding to molecules by hydrophobic interaction. The hydrophobic groups are the portions of the molecule that mainly made up of hydrogen and carbon with minimal amount of oxygen and nitrogen. In the carrier of the present invention a portion of the carrier may have hydrophobic group and other portion of the carrier have poly-cationic moiety and together may provide both hydrophobic and ionic interactions with the load molecule that contains anionic and hydrophobic groups. It is also understood that the hydrophobic group counts as a separate entity from the polymeric backbone, such that, for example, when the polymeric backbone is a polyamino acid, the natural R group on the polyamino acid is not counted as a hydrophobic group in the context of the present invention. For example, a hydrophobic group may be added to a polylysine backbone or poly-cationic group through amide formation from an amine group. Such amide bond formation with amino group of cationic moiety requires that the starting alkyl group or hydrophobic group be alkyl acid or fatty acids containing 4 to 36 carbons.

In a one embodiment, the hydrophobic group comprises alkyl group. In one embodiment, the alkyl group comprises an un-branched alkyl group. In another embodiment, the un-branched alkyl group comprises a double bond. In another embodiment the alkyl group comprises a branched alkyl group. In another embodiment the branched alkyl group comprises a double bond. In another embodiment, the alkyl group comprises a methyl, ethyl or propyl group. In another embodiment the alkyl group is a butyl, pentyl or hexyl group. In another embodiment, the alkyl group is CH₃(CH₂)ₙCO—, —OC(CH₂)ₙCH₂—, —OC(CH₂)ₙCH₂ NH—, —OC(CH₂)ₙCO—, —OC(CH₂)ₙCH₂O—, —OC(CH₂)ₙCH₂S—, —HNC(CH₂)CH₂—, —HNC(CH₂)ₙCO—, —OCH₂(CH₂)ₙCH₂—, or —OCH₂ (CH₂)ₙCO—; wherein “n” is 1-36 inclusive, 4-36 inclusive, 8-24 inclusive, 12, 16, 18, 22, 24 or 36. In another embodiment, the hydrophobic groups comprise an aromatic ring compound. In yet another embodiment the hydrophobic groups comprise a fatty acid, or a fatty acid derivative, such as an alkyl acyl where the number of carbons is from 4-36 inclusive. In another embodiment, the aromatic ring is phenyl. In another embodiment, the aromatic ring is naphthyl. In a further embodiment, the aromatic ring compound is cholesterol.

In one embodiment, hydrophobic functional groups may comprise hydrophobic alkyl groups, which have a general formula [CxHyOz] where x is 1-36; y is 3-71; z is 0-4. It is possible to have z=0 if alkyl halide is the starting material for attaching alkyl group to the amino group poly-cationic moiety. In one embodiment where the alkyl group with x>2, z=1, which is the minimum required for amide bond with the amino group of the poly-cationic moiety. The starting molecules however may have z>1 prior to amide bond formation. The hydrophobic functional groups may also comprise two ended hydrophobic alkyl groups with one end attached to the poly-cationic moiety, which have a general formula [—OC(CH₂)ₓCO—] or [—OC(CH₂)ₓCN—] where x is 2-36, and may further comprise a protective group, analog or derivative thereof covalently attached to the other end of the hydrophobic group.

The chemical link of hydrophobic functional groups to the carrier comprises amide bond to the amino group of the poly-cationic moiety pendant to the polymeric backbone. The poly-cationic moiety is the amide bonded of a hydrophobic functional group comprising an alkyl acyl derived from fatty acids, or aromatic alkyl acyl derived from aromatic alkyl acids, which has a general formula [CxHyOz] where x is 2-36; y is 3-71; z is 1-4. It is preferable that z=1, which is the minimum required for amide bond with the amino group of the poly-cationic moiety. The starting molecules however may have z greater than 1 prior to amide bond formation.

In specific embodiments, each hydrophobic group has molecular weight of 15-700 Daltons inclusive.

Targeting Moieties

The term “targeting moiety,” “targeting molecules,” or “targeting group” refers to any molecular structure which assists the construct of the composition in localizing at a particular target area, entering a target cell(s), and/or binding to a target receptor. In certain embodiments, the composition may further include a targeting group. For example, lipids (including cationic, neutral, and steroidal lipids), antibodies, antibody fragments, chimeric antibodies, lectins, ligands, receptor ligands, sugars, saccharides, steroids, hormones, nutrients, peptides, proteins, enzymes, quasi substrates of enzymes, cell-surface-binding compounds, and extracellular-matrix-binding compounds may serve as targeting moieties. In certain embodiments the targeting group is covalently linked to the distal end the protective group. In other embodiments the targeting group is linked to the terminus of the polymeric backbone.

Chemical Assembly of the Carrier

Attaching protective chains to a polymeric backbone containing amino groups: The present disclosure relates to a polymeric backbone further comprising a protective chain and poly-cationic moiety. In specific embodiments, the modification of a polymeric backbone containing amino groups is the amide covalent attachment of protective chains comprising methoxypolyethyleneglycol. A non-limiting example of an amino group modification along the polymeric backbone is attachment of protective chains comprising acyl polymethoxyoxyethyleneglycol. An example of a protective chain which is not intended to limit the scope of this invention is an acyl PEG, analog, or derivative thereof which can be represented by formula: —CO(CH₂)ₙCOOCH₂CH₂-A-OR₃ or —COCH₂-A-OR₃, where n is 2-22; A is [OCH2CH2]ₓ or [OCH2CH2]ₓ or [OCHCH₃CH₂]ₓ, where x is 17-250, or various combinations of [OCH2CH2], [OCH2CH2], and/or [OCHCH₃CH₂] with total of 17-250 units, R₃ is H, (CH₂)ₚCH₃ or (CH₂)ₚCOOH, and p is 0-7.

In specific embodiments, the modification of a polymeric backbone containing amino groups is the amide covalent attachment of protective chains. An object of the present invention is to provide a method of attaching protective chains to the amino group containing polymeric backbone. The modifications can be done by amide bond formation. As an example that is not intended to limit the scope of this invention, the carboxyl containing protective chain can be attached to the amino group of the polymeric backbone using carbodiimide containing reagent such a 1-ethyl-3-(3-dimethylaminopropyl)-carbodiimide or dicyclohexylcarbodiimide. A carbodiimide reagent contains a functional group consisting of the formula N═C═N. During the process of coupling reaction, the activated carboxyl group O-acylisourea-intermediate can be stabilized by forming N-hydroxysuccinimide ester using N-hydroxysuccinimide. This relatively stable intermediate can react with the amino group of carrier such as polylysine or chitosan to form amino-acyl bond or amide bond. Similar results can also be accomplished by reacting aldehyde containing protective group to the amino group along the carrier. The aldehyde can react with the amino group of carrier such as polylysine or chitosan to form an amino-acyl bond or an amide bond.

Attaching protective chains to a polymeric backbone containing carboxyl groups: The present disclosure relates to a polymeric backbone further comprising a protective chain and poly-cationic moiety. The modification of the polymeric backbone containing carboxyl groups is the amide covalent attachment of an amino group containing protective chains comprising amino polymethoxyoxyethyleneglycol. As an example that is not intended to limit the scope of this invention, the protective chain can be an amino PEG which can be represented by formula —NH(CH₂)ₙNHCOCH₂-A-OR₃, —NH(CH₂)ₙNHCO(CH₂)ₙCOOCH₂CH₂-A-OR₃, where n is 2-22; A is [OCH₂CH₂]ₓ, or [OCH₂CH₂]ₓ or [OCHCH₃CH₂]ₓ, where x is 17-250, or various combinations of [OCH₂CH₂], [OCH₂CH₂], and/or [OCHCH₃CH₂] with total of 17-250 units, R₃ is H, (CH₂)ₚCH₃ or (CH₂)ₚCOOH, and p is 0-7.

Another object of the present invention is to provide methods of attaching protective chains to the polymeric backbone. These modifications can be done by amide bond formation. As an example that is not intended to limit the scope of this invention, the carboxyl group of the polymeric backbone can be activated to react with amino functional group of the protective chains. The activation can be accomplished using carbodiimide containing reagent such a 1-ethyl-3-(3-dimethylaminopropyl)-carbodiimide or dicyclohexylcarbodiimide. A carbodiimide reagent contains a functional group consisting of the formula N═C═N. During the process of activation the carboxyl group forms O-acylisourea-intermediate that can be stabilized by N-hydroxysuccinimide to form N-hydroxysuccinimide ester. This relatively stable intermediate can react with the amino group of protective molecules. If the protective group or molecule that needs to be introduced into the carrier does not have amino group, the amino group can be introduced to this molecule very easily and this process is well known to those skilled in the art.

Attaching protective chains to a polymeric backbone containing hydroxyl groups: The present disclosure relates to a polymeric backbone further comprising a protective chain and poly-cationic moiety. The modification of the polymeric backbone containing hydroxyl groups is the ester or ether bond formation with protective chains comprising methoxypolyethyleneglycol. The modification of hydroxyl groups of polymeric backbone is by ester bond formation with protective groups comprising acyl methoxypolyethyleneglycol. As an example that is not intended to limit the scope of this invention, the protective group can be a PEG with acyl or carbonyl represented by —CO and attached to 0 of hydroxyl group of carrier to form ester. The acyl PEG or its derivative can be represented by formula —CO(CH₂)ₙNHCOCH₂-A-OR₃, —COCH₂CH₂-A-OR₃, or —COCH₂-A-OR₃, where n is 2-22; A is [OCH₂CH₂]ₓ or [OCH₂CH₂]ₓ or [OCHCH₃CH₂]ₓ, where x is 17-250, or various combinations of [OCH₂CH₂], [OCH₂CH₂], and/or [OCHCH₃CH₂] with total of 17-250 units, R₃ is H, (CH₂)ₚCH₃ or (CH₂)ₚCOOH, and p is 0-7.

The modification of a hydroxyl group can be facilitated by the synthesis of acyl halides of protective chains. Synthesis of acyl halides can be done by reaction of the carboxylic acid moiety of protective chains with dichlorosufoxide (SOCl2) or other reagents known to those skilled in the art. The resulting acyl halides are reactive to alcohols including serine, threonine, and tyrosine residue of poly amino acids. The reaction will result in an ester bond formation essentially attaching the protective groups or molecules into the carrier. PEG-epoxide, PEG-isocyanate, PEG-PNC (PEG-nitrophenylcarboxyester) are the PEG analogs that may be used to modify the hydroxyl groups forming ether, ester, and urethane linkage respectively between the protective group and the carrier.

Attaching a poly-cationic moiety to polymeric backbone containing amino groups: The present disclosure relates to a polymeric backbone further comprising a protective chain and poly-cationic moiety. Once the polymeric backbone contains protective chains, the polycationic moiety can be attached by first modifying the remaining amino groups of the polymeric backbone into a carboxyl-containing group such as but not limited to reaction with succinic-anhydride or other anhydride containing molecules. Once the amino groups have been converted to carboxyl groups, the carboxyl groups can be activated to react with poly-cationic molecules such as polyethyleneimine, putrescene, spermine, spermidine, cadaverine, polylysine, polyarginine, and derivatives thereof. Another object of the present invention is to provide a method of attaching a poly-cationic moiety to the polymeric backbone with amino groups along its length. As an example that is not intended to limit the scope of this invention, the modifications can be done by amide bond formation with an anhydride molecule followed by another amide bond formation with a poly-cationic molecule. The resulting carboxyl-containing polymeric backbone can be attached to the amino group of the poly-cationic moiety using a carbodiimide containing reagent such a 1-ethyl-3-(3-dimethylaminopropyl)-carbodiimide or dicyclohexylcarbodiimide. A carbodiimide reagent contains a functional group consisting of the formula N═C═N. During the process of coupling reaction, the activated carboxyl group (O-acylisourea-intermediate) can optionally be stabilized by forming N-hydroxysuccinimide ester using N-hydroxysuccinimide. This relatively stable intermediate can react with the amino group of the poly-cationic moiety to form amino-acyl bond or amide bond.

Attaching a poly-cationic moiety to polymeric backbone containing carboxyl groups: The present disclosure relates to a polymeric backbone further comprising a protective chain and poly-cationic moiety. Once the polymeric backbone contains protective chains, the poly-cationic moiety can be attached to the carboxyl groups by activating the carboxyl groups to react with poly-cationic molecules such as polyethyleneimine, putrescene, spermine, spermidine, cadaverine, polylysine, polyarginine, and derivatives thereof. Another object of the present invention is to provide a method of attaching poly-cationic moiety to the polymeric backbone with carboxyl groups along its length. As an example that is not intended to limit the scope of this invention, the modifications can be done by amide bond formation with poly-cationic molecule. The carboxyl containing polymeric backbone can be attached to the amino group of the poly-cationic moiety using a carbodiimide containing reagent such a 1-ethyl-3-(3-dimethylaminopropyl)-carbodiimide or dicyclohexylcarbodiimide. A carbodiimide reagent contains a functional group consisting of the formula N═C═N. During the process of coupling reaction, the activated carboxyl group (O-acylisourea-intermediate) can optionally be stabilized by forming N-hydroxysuccinimide ester using N-hydroxysuccinimide. This relatively stable intermediate can react with the amino group of the poly-cationic moiety to form amino-acyl bond or amide bond.

Attaching a poly-cationic moiety to a polymeric backbone containing hydroxyl groups: The present invention relates to a polymeric backbone further comprising protective chains and poly-cationic moiety. Once the polymeric backbone contains protective chains, the poly-cationic moiety can be attached by first modifying the remaining hydroxyl groups of the polymeric backbone into a carboxyl containing group such as but not limited to reaction with succinic-anhydride or other anhydride containing molecules. Once the hydroxyl groups have been converted to carboxyl groups, the carboxyl groups can be activated to react with poly-cationic molecules such as polyethyleneimine, putrescene, spermine, spermidine, cadaverine, polylysine, polyarginine, and derivatives thereof. Another object of the present invention is to provide a method of attaching a poly-cationic moiety to the polymeric backbone with hydroxyl groups along its length. As an example that is not intended to limit the scope of this invention, the modifications can be done by ester bond formation with anhydride molecule followed by amide bond formation with poly-cationic molecule. After modification of hydroxyl groups to carboxyl groups, the new carboxyl group polymeric backbone can be attached to the amino group of the poly-cationic moiety using a carbodiimide containing reagent such a 1-ethyl-3-(3-dimethylaminopropyl)-carbodiimide or dicyclohexylcarbodiimide. A carbodiimide reagent contains a functional group consisting of the formula N═C═N. During the process of coupling reaction, the activated carboxyl group (O-acylisourea-intermediate) can optionally be stabilized by forming N-hydroxysuccinimide ester using N-hydroxysuccinimide. This relatively stable intermediate can react with the amino group of the poly-cationic moiety to form amino-acyl bond or amide bond.

Attaching hydrophobic groups to a poly-cationic moiety of a polymeric backbone: The present invention relates to a polymeric backbone further comprising protective chains pendant to the backbone; a poly-cationic moiety pendant to the backbone, and a hydrophobic group covalently linked to the poly-cationic moiety. The hydrophobic group can be attached to the amino groups of a poly-cationic moiety. A hydrophobic group can come from fatty acid (C4-C22) anhydride. For example, reaction of the poly-cationic amino groups with palmitic acid anhydride forms a long chain hydrophobic group comprising 16 carbons. Most fatty acid anhydrides may be used in this fashion. Alternatively, the hydrophobic group can be introduced as a N-hydroxy succinimide ester which will react readily with the amino groups of the poly-cationic moiety of the carrier. The activation of the carboxyl group of the hydrophobic group can be accomplished using carbodiimide containing reagent such a 1-ethyl-3-(3-dimethylaminopropyl)-carbodiimide or dicyclohexylcarbodiimide. A carbodiimide reagent contains a functional group consisting of the formula N═C═N. During the process of activation, the carboxyl group forms O-acylisourea-intermediate that can optionally be stabilized by N-hydroxysuccinimide to form N-hydroxysuccinimide ester. This relatively stable intermediate can react with the amino group of hydrophobic groups. If the hydrophobic group or molecule does not have an amino group, an amino group can be introduced to the molecule and the chemistry is well known to those skilled in the art.

Another object of the present invention is to provide a method of attaching a hydrophobic group to the poly-cationic moiety of the carrier. The modification of an amino group of a poly-cationic moiety can be facilitated by synthesis of acyl halide of fatty acids, carboxyl aromatic hydrocarbons, or dicarboxylic alkyl. Synthesis of acyl halides can be done by reaction of the carboxylic acid moiety with dichlorosulfoxide (SOCl₂) or other reagents known to those skilled in the art. The resulting acyl halides are reactive to amino functional groups present in the poly-cationic moiety. The reaction will result in amide bond formation attaching the hydrophobic groups or molecules to the poly-cationic moiety.

The type of chemical link to use in attaching the poly-cationic moiety and protective groups will depend on the desired biological half-life of the complex and the therapeutic agent associated with the complex. In certain embodiments, if a longer half-life in biological tissue and/or fluid is desired, amide bonds can be utilized. In alternative embodiments, where a shorter half-life is desired, ester bonds can be used. In particular embodiments, mixtures of both chemical bonds can be used to achieve the desired stability for a specific therapeutic agent to be delivered. The S—S bond may be used to achieve a desired property of the carrier that would be beneficial for its intended therapeutic and diagnostic purpose.

Methods of Use

The novel compositions disclosed herein can be selected for use in methods of treatment of patients according to the combinations of carriers provided and the underlying disease or physiologic condition of the patient and/or the molecular target and its location. The cationic core based pharmaceutical compositions can be administered by any suitable means or route in a pharmaceutically acceptable carrier, including parenteral, transdermal, rectal, intrapulmonary, and intranasal, and, if desired, for local injection. Parenteral administration routes include intramuscular, intravenous, intra-arterial, intraperitoneal, or subcutaneous administration.

The appropriate dosage of the cationic core composition will depend on the type of disease or condition to be treated, the severity and course of the disease, the patient's clinical history and response to the cationic core composition, and the discretion of the attending physician. Cationic core compositions can suitably be administered to the patient in a single dose, in divided doses, or over a series of treatments. Also, the present invention contemplates mixtures of more than one cationic core composition, as well as use in combination with other therapeutic agents.

In certain embodiments, the dosage of the subject compounds will generally be in the range of about 0.01 ng to about 1 g per kg body weight, specifically in the range of about 1 ng to about 0.1 g per kg, and more specifically in the range of about 100 ng to about 10 mg per kg.

The cationic core composition will be administered to a patient in need in a therapeutically effective amount. A therapeutically effective amount refers to the amount of composition that will provide a therapeutic benefit to the patient. In certain embodiments, the term refers to an amount of the therapeutic agent that, when loaded to the cationic core composition of the present invention and administered to the patient, produces some desired effect at a reasonable benefit/risk ratio applicable to any medical treatment. In certain embodiments, the term refers to that amount necessary or sufficient to eliminate, reduce or maintain (e.g., prevent the spread of) a tumor or other target of a particular therapeutic regimen. The effective amount may vary depending on such factors as the disease or condition being treated, the particular constructs being administered, the size of the subject and/or the severity of the disease or condition. One of ordinary skill in the art may empirically determine the therapeutically effective amount of a particular compound without necessitating undue experimentation. In certain embodiments, the term refers to that amount necessary or sufficient for a use of the subject compositions described herein. By way of example, in the treatment of obesity, the therapeutically effective amount is the amount of composition of the present invention with corresponding load molecule(s) such as, but not limited to, anti-ghrelin siRNA that will reduce ghrelin, appetite, and weight. By way of another example, in the treatment of obesity, the therapeutically effective amount is the amount of composition of the present invention with corresponding load molecule(s) such as, but not limited to, anti-Neuropeptide Y (NPY) siRNA that will reduce NPY, appetite, and weight. In the treatment of obesity, the therapeutically effective amount is the amount of composition of the present invention with corresponding load molecule(s) such as, but not limited to, RNA-oligonucleotide-linked leptin that will increase leptin, decrease appetite and weight. In the treatment of obesity, the therapeutically effective amount is the amount of composition of the present invention with corresponding load molecule(s) such as, but not limited to, RNA-oligonucleotide-linked PYY that will increase PYY, decrease appetite and weight. By way of another example, in the treatment of insulin-insufficient diabetes, the therapeutically effective amount is the amount of composition of the present invention with corresponding load molecule(s) that will improve glucose homeostasis or normalize blood glucose level of the patient and/or regenerate the beta-islet cells in the pancreas. The regeneration of the beta-islet cells can be indirectly measured by monitoring blood glucose level, Hemoglobin Alc level, C-peptide level, or insulin level in the blood.

The cationic core compositions will be formulated, dosed, and administered in a fashion consistent with good medical practice. Factors for consideration in this context include the particular disorder being treated, the particular mammal being treated, the clinical condition of the individual patient, the cause of the disorder, the site of delivery of the peptide, the method of administration, the scheduling of administration, and other factors known to medical practitioners. The therapeutically effective amount of cationic core composition to be administered will be governed by such considerations, and is the minimum amount necessary to prevent, ameliorate, or treat a disease or disorder.

The precise time of administration and dosage of any particular compound that will yield the most effective treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation or will consist of monitoring the subject and adjusting the dosage and/or timing. An effective dose or amount, and any possible affects on the timing of administration of the dose, may need to be identified for any particular composition of the present invention. Dosages for the compounds of the present invention may be readily determined by techniques known to those of skill in the art. In one embodiment, the effective dose may be determined by routine experiment using one or more groups of animals (preferably at least 5 animals per group), or in human trials if appropriate. In another embodiment, the effectiveness of the composition and method of treatment or prevention may be assessed by administering the cationic core composition and assessing the effect of the administration by measuring one or more indices associated with the disease or condition of interest, and comparing the post-treatment values of these indices to the values of the same indices prior to treatment. For repeated administrations over several days or longer, depending on the condition, the treatment is sustained until a desired suppression of disease symptoms occurs. Accordingly, the method of treatment embodiments can include obtaining single or sequential blood or other body fluid samples from a patient after administration of the composition and quantitatively assaying for the free peptide by use of assays known in the art; e.g., HPCL, bioassay, mass spectroscopy and the like. Resulting values can be compared to threshold values known in the art to correspond to therapeutically-effective concentrations; e.g., area under the curve (AUC), half-life, Cmax, and other pharmacokinetic parameters known in the art.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXEMPLIFICATION

Overview of the Synthetic Method: Poly-cationic core carriers of the present invention include a central polymeric backbone, a poly-cationic moiety, a protecting group, and, optionally hydrophobic group and/or a targeting group. Each group is linked together covalently and the poly-cationic moiety group is capable of forming reversible binding (based on ionic interactions) with an anionic load molecule (therapeutic or diagnostic agent) such as polynucleotides (DNA, RNA), anionic peptides/proteins, anionic drugs and derivatives thereof. The reversible linkage between the carrier and a load molecule includes ionic interactions between the anionic load molecule and the poly-cationic moiety of the carrier.

The synthesis of a cationic-core carrier load molecule complex from a polymeric carrier containing amino, carboxyl, hydroxyl groups, or thiol groups generally involves three synthetic steps: 1) covalent modification of a backbone carrier to add protective chains; 2) modification of the product from step 1 to add a poly-cationic moiety; and 3) incubating the product from step 2 with an anionic load molecule, such as, for example, incubation with siRNA to achieve formation of a poly-cationic core carrier-siRNA complex.

### Example 1

Synthesis of MPEG-poly-L-lysine (5000; 40,000; 73%; 40PLPEG573): The reagents, MPEG-succinimidyl-succinate and polylysine, are commercially available and their syntheses are well known in the art. Poly-L-lysine (200 mg; Polylysine Hydrobromide; Sigma chemical Co.; DPvis:264; MWvis: 55,200; DPmalls:190; MWmalls:39,800; 0.7 mmoles aminogroup by TNBS assay Sparado et al. Anal Biochem 96:317, 1979) was dissolved in 10 ml of 0.1 M carbonate buffer pH 8.35 and 1150 mg of MPEG-succinimidyl-succinate was added, vortexed, and incubated overnight at room temperature. The next day, aliquots were taken and the amount of amino groups remaining was quantified using trinitrobenzenesulfonic acid (Sparado et al. Anal Biochem 96:317, 1979). The result indicated that 73% of amino group had been conjugated to MPEG. To cap the carboxyl terminal of polylysine that can potentially interfere with the next reaction, 600 ul of ethylenediamine and 100 mg EDC was added mixed and incubated at room temperature for 1 hr. The solution (200 ml) was washed by filtration through 100 kDa cut-off filter membrane (GE-Amersham Biosciences Corp, Westborough, Mass.) with five changes of water. The resulting PLPEG complex was lyophilized and weighed giving a yield of 860 mg. The resulting product has an estimated MW of 730 kDa based on the number of amino groups that had been derivatized by MPEG. The number of free amino groups per mg of final product was 430 nmoles/mg. It should be noted that if MPEG-succinimidyl-succinate used is contaminated with free succinate, which is quite common, the amount of PEG will be less than what is expected from the amino group analysis and will be inconsistent with the amount of amino group per mg of final product.

### Example 2

Synthesis of MPEG-poly-L-lysine (5 kDa PEG; 40 kDa PL; 55% saturation of amino groups; 40PLPEG555): The reagents, MPEG-succinimidyl-succinate and polylysine, are commercially available and their syntheses are well known in the art. Poly-L-lysine (200 mg; Polylysine Hydrobromide; Sigma chemical Co.; DPvis:264; MWvis: 55,200; DPmalls:190; MWmalls:39,800; 0.7 mmoles aminogroup by TNBS assay Sparado et al. Anal Biochem 96:317, 1979) was dissolved in 10 ml of 0.1 M carbonate buffer pH 8.35 and 900 mg of MPEG-succinimidyl-succinate was added, vortexed, and incubated overnight at room temperature. The next day, aliquots were taken and the amount of amino groups remaining was quantified using trinitrobenzenesulfonic acid (Sparado et al. Anal Biochem 96:317, 1979). The result indicated that 55% of the amino groups had been conjugated to MPEG. To cap the carboxyl terminal of polylysine that can potentially interfere with the next reaction, 600 ul of ethylenediamine and 100 mg EDC was added mixed and incubated at room temperature for 1 hr. The solution (200 ml) was washed by filtration through 100 kDa cut-off filter membrane (GE-Amersham Biosciences Corp, Westborough, Mass.) with five changes of water. The resulting PLPEG complex was lyophilized and yielded of 860 mg. The resulting product has an estimated MW of 560 kDa based on the number of amino groups that had been derivatized by MPEG. It should be noted that if MPEG-succinimidyl-succinate used is contaminated with free succinimidyl-succinate, the amount of PEG will be less than what is expected from the amino group analysis and will be inconsistent with the amount of amino group per mg of final product.

### Example 3

Synthesis of MPEG-poly-1-lysine (5 kDa PEG; 40 kDa PL; 22% saturation of amino groups; 40PLPEG522): The reagents, MPEG-succinimidyl-succinate and polylysine, are commercially available and their syntheses are well known in the art. Poly-L-lysine (200 mg; Polylysine Hydrobromide; Sigma chemical Co.; DPvis:264; MWvis: 55,200; DPmalls:190; MWmalls:39,800; 0.7 mmoles aminogroup by TNBS assay Sparado et al. Anal Biochem 96:317, 1979) was dissolved in 10 ml of 0.1 M carbonate buffer pH 8.35 and 600 mg of MPEG-succinimidyl-succinate was added, vortexed, and incubated overnight at room temperature. The next day, aliquots were taken and the amount of amino groups remaining was quantified using trinitrobenzenesulfonic acid (Sparado et al. Anal Biochem 96:317, 1979). The result indicated that 22% of amino groups had been conjugated to MPEG. The solution (200 ml) was washed by filtration through 100 kDa cut-off filter membrane (Amersham Biosciences Corp, Westborough, Mass.) with five changes of water. The resulting PLPEG complex was lyophilized and weighed giving a yield of 320 mg. The resulting product has an estimated Mw of 250 kDa based on the number of amino groups that had been derivatized by MPEG. It should be noted that if MPEG-succinimidyl-succinate used is contaminated with free succinimidyl-succinate, the amount of PEG will be less than what is expected from the amino group analysis and will be inconsistent with the amount of amino group per mg of final product.

### Example 4

Synthesis of MPEG-polylysine (5 KDa PEG; 40 kDa PL; 9% saturation of amino groups; 40PLPEG509): The reagents, MPEG-succinimidyl-succinate and polylysine, are commercially available and their syntheses are well known in the art. Poly-L-lysine (200 mg; Polylysine Hydrobromide; Sigma chemical Co.; DPvis:264; MWvis: 55,200; DPmalls:190; MWmalls:39,800; 0.7 mmoles aminogroup by TNBS assay Sparado et al. Anal Biochem 96:317, 1979) was dissolved in 10 ml of 0.1 M carbonate buffer pH 8.35 and 300 mg of MPEG-succinimidyl-succinate was added, vortexed, and incubated overnight at room temperature. The next day, aliquots were taken and the amount of amino group remaining was quantified using trinitrobenzenesulfonic acid (TNBS) (Sparado et al. Anal Biochem 96:317, 1979). The result indicated that 9% of amino groups had been conjugated to MPEG. The solution (200 ml) was washed by filtration through 100 kDa cut-off filter membrane (Amersham Biosciences Corp, Westborough, Mass.) with five changes of water. The resulting PLPEG complex was lyophilized and weighed giving a yield of 300 mg. The resulting product has an estimated MW of 125 kDa based on the number of amino groups that had been derivatized by MPEG. It should be noted that if MPEG-succinimidyl-succinate used is contaminated with free succinimidyl-succinate, the amount of PEG will be less than what is expected from the amino group analysis and will be inconsistent with the amount of amino group per mg of final product.

### Example 5

Synthesis of 40PLPEG537PEI12 (lot#20070926): One gm of 40 PL (Sigma P3995; this time 1 gm has 2.5 mmol NH2) was dissolved in 50 ml of 200 mM HEPES. 3.5 g of MPFGsuccinate (0.7 mmol) in 25 ml of 10 mM MES pH=4.7 was activated by adding 175 mg of NHSS (N-Hydroxysulfosuccinimide; Mw=217.14; 0.8 mmol), followed by 350 mg EDC (1-ethyl-3-(3-dimethylaminopropyl)carbodiimide; Mw=191.71; 1.8 mmol). Activation is allowed to proceed for 20 minutes. The activated MPEGsuccinate was added to 40 PL solution and allowed to react. After 2 hrs, additional 3.5 g of MPEGsuccinate was activated and added as above and allowed to react overnight. The next day amino group was measured and found to be 1.5 mmol indicating 40% saturation of amino group. The sample was lyophilized (13 g) without cleaning and stored at 4° C. The following week the material was dissolved in 37 ml water, 2 g Succinic anhydride (20 mmol) was added and 200 ul TEA added followed by titration (200 ul at a time) to pH 7.5-8.0 using 10M NaOH. After 2 hours, the amino group was measured by taking 15 ul and diluting to 1 ml (67 fold; giving 0.2 mg/ml equivalent of original PL) and result shows that no amino group remains. The material was washed with 20 volumes with water using ultrafiltration cartridge with molecular weight cut off (MWCO) of 10 La (UFP-100-E-5A; GE Healthcare). The 40PLPEG537-succinate was dried (5.9 g) and 2.95 g (0.9 mmol carboxyl each) was made up in 10 ml of 10 mM MES and activated by adding 250 mg of NHSS (mw=217.14; 1.15 mmol, followed by 500 mg EDC (mw=191.71; 2.6 mmol). Activation was allowed to proceed for 20 minutes. Fifty ml of PEI12 (Sigma Cat#482595; 50% PEI Mw 1200 or 20 mmol) was prepared in 50 ml of 1 M HEPES buffer with pH adjustment to 8.0 using approximately 50 ml of 6 N HCl. Activated 40PLPEG537-succinate solutions above were added to the PEI12 solution. After a 2 hour reaction, PEI 12 reaction was washed with 25 volumes of water using ultrafiltration cartridge with molecular weight cut off (MWCO) of 10 kDa (UFP-100-E-5A; GE Healthcare). PEI wash solution was tested for amino groups using TNBS until clear (50 volumes of water). The products were lyophilized yielding 3.1 g of 40PLPEG37PEI12; lot#20070926. The amino groups was measured by TNBS and found to be 553 mmol/mg. The molecular diameter of this material was 19 nm as measured by GPC (column 0.78×30 cm; Tosoh G4000WXL; with PBS/15% Acetonitrile mobile phase flowing at 0.6 ml/min).

### Example 6

Synthesis of 40PLPEG537DAPEI4 (Lot#20071005): A 40 PL solution was prepared by dissolving 1 gm of 40 PL (Sigma P3995; this time 1 gm has 2.5 mmol NH2) in 50 ml of 200 mM HEPES buffer, pH 7.3. MPEGCM (3.5 grams; MethoxyPEGCarboxymethyl; 0.7 mmol; Mw=5 kDa; Sigma Chemical Co. Cat#70718; Fluka lot#1373341) was dissolved in 25 ml of 10 mM MES pH=4.7 and activated by adding 175 mg of NHSS(N-Hydroxysulfosuccinimide; Mw=217.14; 0.8 mmol), followed by 350 mg EDC (1-ethyl-3-(3-dimethylaminopropyl)carbodiimide; Mw-191.71; 1.8 mmol). After 20 minutes, the activated MPEGCM (2×3.5 grams) was added to 40 PL solution. After 2 hours, another 3.5 grams of MPEGCM was activated in a similar way and added to the 40 PL solution. Analysis of amino groups in the 40 PL solution by TNBS assay showed a reduction in amino group (1.5 mmol) achieving 40% MPEG saturation. Triethylamine or TEA (200 ul) was added followed by 6 grams Succinic anhydride (SA). The pH of the solution was adjusted by titration (200 ul at a time) to pH 7.5-8.0 using 10M NaOH. After an hour, additional 2 grams of SA was added, the pH adjusted to 8.0 and 1 hr later the amino group was measured by taking 150 ul diluting to 4 ml water for TNBS amino group analysis. The amino group was found to be 0 uM. The sample was washed with 20 volumes of water using ultrafiltration cartridge with molecular weight cut off (MWCO) of 10 kDa (UFP-100-E-5A; GE Healthcare) and lyophilized (6 grams). The size of this material was 20 nm as measured by GPC (column 0.78×30 cm; Tosoh G4000WXL; with PBS/15% Acetonitrile mobile phase flowing at 0.6 ml/min). Twenty ml of PIE4 (400 Da PEI or PEI4; 50 mmol; Sigma Chem. Co. Cat#468533; Batch#115EH) was neutralized with 40-50 ml 6NHCl and buffered with 20 ml of 1 M HEPES at pH 7.5-8.0 ending up with total volume of about 100 ml. The 40PLPEG537DA-succinate was dissolved in 100 ml of 20 mM MES and 500 mg of NHSS was added followed by 1 g EDC and stirred for 20 minutes to activate. The activated 40PLPEG537DA-succinate was added to PEI4 solution and stirred for 6 hrs. Reaction mixture was washed with 30 volumes of water using ultrafiltration cartridge with molecular weight cut off (MWCO) of 10 kDa (UFP-100-E-5A; GE Healthcare) and lyophilized (5.1 g; 40PLPEG537PEI4; Lot#20071005). Amino group was measured (2 mg/ml, 200 ug/ml, 100 ug/ml, 2 ug/ml, and 1 ug/ml). The amino group content prior to succinylation and PEI4 addition is 0.200 umol/mg. There is 79 uM in 200 ug/ml which is 395 nmol/mg. The size of this material was 19 nm as measured by GPC (column 0.78×30 cm; Tosoh G4000WXL; with PBS/15% Acetonitrile mobile phase flowing at 0.6 ml/min). This material was used in experiment outlined below.

### Example 7

Synthesis of 20PLPEG1055DA-PEI4 (Lot#20080415): A 20 PL solution was prepared by dissolving 2 gm of 20 PL (20 kDa polylysine from Sigma Chemical Co; Q4926 SAFC lot# 018K7775; DP=126; 2 gm has 4.60 mmol NH2) in 25 ml of 1M HEPES buffer, pH 7.3. The amino group was measured by taking 6.0 ul and diluting it to 8 ml (0.05 mg20 PL/ml) for TNBS (tirinitrobenzinesulfonic acid) assay. The TNBS assay was adapted from Spadaro et al. Anal. Biochem, 96, 317-321. The amino group was found to be 4.60 mmol NH2/2 g. In a separate container, 20 g of MPEGCM (MethoxyMolyEthyleneGlycolCarboxyMethyl; Mw=10000 Da; 2.0 mmol; SunBright; ME-100HS; lot#M62503) was dissolved in 60 ml of 80% ethanol with 20 mM MES pH=4.7 (1200 ul of 1M MES added to 60 ml) and 500 mg of NHS (4.35 mmol; N-Hydroxysuccinimide; Mw=15.14) was added, once dissolved 2.0 g EDC (mw-191.71; 10.43 mmol) was added while stirring magnetically. Activation was allowed to proceed for 20 minutes and the activated MPEGCM was added directly to 20 PL solution. The pH was adjusted to pH 7.1 slowly with 10N NaOH one drop at a time, and allowed to react for 2 hours. Aliquot (exactly 10 ul per 100 ml solution or 13.9 ul for 139 ml in this case) was taken and diluted to 4 ml water for TNBS amino group analysis. It was found to be 48 uM (1.92 mmol total) indicating 58% PEG saturation. This is the 20PLPEG1055DA solution. Analysis by Gel Permeation Chromatography (GPC) using TosohG4000WXL column (0.79×30 cm) eluted with phosphate buffered saline (PBS; 11.9 mM phosphate, 137 mM NaCl, 2.7mMKPO₄, pH 7.4) containing 15% Acetonitrile at a flow rate of 0.6 ml/min showed a retention time of 11.7 min (approx 22.2 um molecular diameter) and also showing 95% incorporation of MPEG. Succinic Anhydride (2 grams; 20 mmol) was added followed by 200 uL TEA. The reaction was slowly titrated with 10 N NaOH to pH 7.1, stirred for 4 hours. After four hours, the amino groups was measured and found to be 0 uM. GPC analysis using TosohG400WXL column (0.79×30 cm) eluted with phosphate buffered saline (PBS; 11.9 mM phosphate, 137 mM NaCl, 2.7mMKPO₄, pH 7.4) containing 15% Acetonitrile at a flow rate of 0.6 ml/min showed a retention time of 11.7 min or approx 22.2 nm molecular diameter. The reaction mixture was washed using 100 kDa MWCO filter and lyophilized (13.1 grams). PEI4 (25 ml 400 Da PEI or PEI4; 62 mmol; Sigma Chem. Co. Cat#458533; Batch#0115EH) was mixed with 25 ml of 1M HEPES buffer and adjusted to pH7.4 with 40 mL of 6N HCl. 20PLPEG1055DA-SA (6.9 g; 1.0 mmol carboxyl) was dissolved in 30 ml of 20 mM MES and 230 mg NHS (mw 115.09; 2 mmol) was added, followed by 1000 mg EDC (mw=191.71; 5.2 mmol) and the pH was maintained to 4.7 by HCl during the minute reaction. After 20 minutes the solution was added to solution in PEI4 solution. After 2 hours, the reaction mixture was washed with at 20 volumes of water using ultrafiltration cartridge with molecular weight cut off (MWCO) of 100 kDa (UFP-100-E-5A; GE Healthcare) and lyophilized yielding 6.0 grams of materials. GPC analysis using TosohG4000WXL column (0.79×30 cm) eluted with phosphate buffered saline (PBS; 11.9 mM phosphate, 137 mM NaCl, 2.7mMKPO₄, pH 7.4) containing 15% Acetonitrile at a flow rate of 0.6 ml/min showed a retention time of 11.6 min or approx 23.1 nm in diameter. Sample 20PLPEG1055DAPEI4 was filtered sterilized using 0.2 um filter (polysulfone) and lyophilized yielding 2.0 grams (lot#20080415). One mg/ml was analyzed and contained 186+/−5 nmol amino group/mg.

### Example 8

Synthesis of 20PLPEG550DA-PEI4 (lot#20080603a): A 20 PL solution was prepared by dissolving 6 grams of 20 PL (20 kDa poly-L-lysine; Sigma Chemical Co. Q4926 SAFC lot# 018K7775; DP=126; 2 gm has 4.72 mmol NH2) in 135 ml of 1 M HEPES buffer, pH 7.3. In a separate container, 45 g of MPEGCM (MethoxyPolyEthyleneGlycolCarboxy-Methyl; Mw=5000; 9.0 mmol; Laysan Bio; lot#108-41) was dissolved in 150 ml of 80% ethanol with 10 mM MES pH=4.7 (1.5 ml of 1M MES added to 150 ml) with 2.25 g of NHS (Mw=115.14; 19.6 mmol), once dissolved 4.5 g EDC (Mw=191.71; 23.5 mmol) was added while stirring. Activation was allowed to proceed for 20 minutes and the activated MPEG-CM was added directly to 20 PL solution. The pH was adjusted to pH 7.1 slowly with 10N NaOH one drop at a time, and allowed to react for 2 hours. An aliquot (exactly 10 ul per 100 ml solution or 11.5 ul in this case 115 ml) was taken and diluted to 4 ml water for amino group analysis by TNBS assay. It was found to be 54 uM (2.16 mmol total) indicating 54% PEG saturation. This is the 20PLPEG550DA solution. GPC analysis of 20PLPEG550DA using TosohG4000WXL column (0.79×30 cm) eluted with phosphate buffered saline (PBS; 11.9 mM phosphate, 137 mM NaCl, 2.7mMKPO₄, pH 7.4) containing 15% Acetonitrile at a flow rate of 0.6 ml/min showed a retention time of 12.8 min (approx 14.4 nm in diameter) and also showing 95% incorporation of PEG. Succinic Anhydride (6 grams; 20 mmol) was added to 20PLPEG550DA solution followed by 600 uL TEA. The reaction was slowly titrated with 10 N NaOH to pH 7.1, stirred for 4 hours. The remaining amino groups was measured after 4 hours and found to be 0 uM indicating that 20PLPEG550DA-SA has been formed. GPC analysis using TosohG400WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 12.3 min or approx 17.6 nm in diameter. The reaction mixture was concentrated to 400 ml and washed with 10 changes of water in a 100,000 MWCO ultrafiltration cartridge (UFP-100-E-5A; GE-Amersham) and lyophilized yielding 31 grams (20PLPEG550DA-SA; lot#20080523). Ten ml PEI4 (400 Da PEI or PEI4; 25 mmol; Sigma Chem. Co. Cat#468533; Batch#0115EH) was dissolved in 20 ml of 1M HEPES and adjusted to pH7.4 with 166 mL of 6N HCl. 20PLPEG550DA-SA (7.7 grams; 1.2 mmol carboxyl) was dissolved in 30 ml of 20 mM MES and 260 mg NHS (Mw=115.09; 2.3 mmol) was added, followed by 1.2 g EDC (Mw-191.71; 6.3 mmol) and pH goes up slowly to 6. During the 20 minute reaction, pH is maintained to 4.7 by HCl. After 20 minutes, activated 20PLPEG550DA-SA solution was added to PEI4 solution. After 2 hours, the pH of the reaction mixture was adjusted to pH 5.0 with 6N HCl and followed by addition of 1.2 g EDC (Mw=−191.71; 6.3 mmol). After 20 min, the pH of the reaction mixture was adjusted hack to pH7.2 with 10N NaOH. The reaction mixture, now containing 20PLPEG550DAPEI4, was loaded into a 100 kDa MWCO ultrafiltration cartridge (UFP-100-E-5A; GE-Amersham), concentrated to 100 ml and washed with 10 volume changes of water. GPC analysis using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.9 min or approx 20.6 nm molecular diameter for the 20PLPEG550DAPEI4. The 20PLPEG550DA-PEI4 product was filtered sterilized using 0.2 um filter (polysulfone; Nalgene, Rochester N.Y.) and lyophilized yielding 7.2 grams (20PLPEG550DAPEI4; lot#20080603a). One mg/ml was analyzed and contain 204+/−5 uM NH2 or 204 nmol/mg.

### Example 9

Synthesis of 20PLPEG550DAPEI8 (lot#20080604a): Twenty ml PEI8 (800 Da PEI or PEI8; 25 mmol; Sigma Chem Co. Cat#408719; Batch#08831KD) was dissolved in 20 ml of 1M HEPES and adjusted to pH7.4 with 32 mL of 6N HCl. 20PLPEG550DA-SA (7.7 grams; 1.2 mmol carboxyl; from Example 8 lot#20080523) was dissolved in 30 ml of 20 mM MES and 260 mg NHS (Mw=115.09; 2.3 mmol) was added, followed by 1.2 g EDC (Mw=191.71; 6.3 mmol) and pH goes up slowly to 6. During the 20 minute reaction, pH is maintained to 4.7 by HCl. After 20 minutes, activated 20PLPEG550DA-SA solution was added to PEI8 solution. After 2 hours, the pH of the reaction mixture was adjusted to pH 5.0 with 6N HCl and followed by addition of 1.2 g EDC (Mw=191.71; 6.3 mmol). After 20 min, the pH of the reaction mixture was adjusted back to pH7.2 with 10N NaOH. The reaction mixture, now containing 20PLPEG550DAPEI8, was loaded into a 100 kDa MWCO ultrafiltration cartridge (UFP-100-E-5A; GE-Amersham), concentrated to 100 ml and washed with 10 volume changes of water. GPC analysis using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.7 min or approx 22.2 um molecular diameter for the 20PLPEG550DAPEI8. The 20PLPEG550DAPEI8 product was filtered sterilized using 0.2 um filter (polysulfone; Nalgene, Rochester N.Y.) and lyophilized yielding 7.6 grams (20PLPEG550DAPEI8; lot#20080604a). One mg/ml was analyzed and contain 304+/−5 uM NH2 or 304 mmol/mg.

### Example 10

Synthesis of 20PLPEG550DAPEI12 (lot#20080605a): Fifty ml PEI12 (800 Da PEI or PEI8; 42 mmol; Sigma Chem Co. Cat#482595; Batch#04418JH) was dissolved in 20 ml of 1M HEPES and adjusted to pH7.4 with 45 mL of 6N HCl. 20PLPEG550DA-SA (7.7 grams; 1.2 mmol carboxyl; from Example 8 lot#20080523) was dissolved in 30 ml of 20 nM MES and 260 mg NHS (Mw=115.09; 2.3 mmol) was added, followed by 1.2 g EDC (Mw=191.71; 6.3 mmol) and pH goes up slowly to 6. During the 20 minute reaction, pH is maintained to 4.7 by HCl. After 20 minutes, activated 20PLPEG550DA-SA solution was added to PEI12 solution. After 2 hours, the pH of the reaction mixture was adjusted to pH 5.0 with 6N HCl and followed by addition of 1.2 g EDC (Mw=191.71; 6.3 mmol). After 20 min, the pH of the reaction mixture was adjusted back to pH7.2 with 10N NaOH. The reaction mixture, now containing 20PLPEG550DAPEI12, was loaded into a 100 kDa MWCO ultrafiltration cartridge (UFP-100-E-5A; GE-Amersham), concentrated to 100 ml and washed with 10 volume changes of water. GPC analysis using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.7 min or approx 22.2 nm molecular diameter for the 20PLPEG550DAPEI12. The 20PLPEG550DAPEI12 product was filtered sterilized using 0.2 um filter (polysulfone; Nalgene, Rochester N.Y.) and lyophilized yielding 7.8 grams (20PLPEG550DAPEI8; lot#20080605a). One mg/ml was analyzed and contain 448+/−5 uM NH2 or 448 nmol/mg.

### Example 11

Synthesis of 20PLPEG550DAPEI4C18 (Lot#2008603b): 20PLPEG550DAPEI4 (2 grams; lot#20080603a, see example 8 above) was dissolved in 8 ml of 1M HEPES with 4 ml of ethanol. An aliquot (4 ul) was taken and diluted to 4 ml for amino group measurement by TNBS and found to be 204 mmol NH₂/mg. The 20PLPEG550DAPEI4 solution was warmed to 50 degrees Celsius and constantly stirred. Stearic acid or C18 fatty acid (132 mg; Mw=284.48; 0.46 mmol) was dissolved in 9 ml ethanol, 1.0 ml of 1M MES pH 4.7 was added followed by 55 mg NHS (Mw=115; 0.48 mmol), and 220 mg EDC (Mw=191.71; 1.15 mmol). Activation was allowed to proceed for 20 minutes. The activated C18 fatty acid was added to 20PLPEG550DAPEI4 solution and allowed to react for an hour. After an hour, the pH was adjusted to pH4.7 with 6N HCl and 220 mg EDC (mw=191.71; 1.15 mmol) was added. After 20 minute reaction, the pH was adjusted back to pH7.1. The reaction mixture was cooled to room temperature and loaded into a 100,000 MWCO ultrafiltration cartridge (UFP-100-E-5A; GE-healthcare-Amersham), concentrated to 100 ml and washed with 10 volume changes of 80% ethanol, followed by 10 volume changes with water. Sample 20PLPEG550DAPEI4C1 was filtered sterilized using 0.2 um filter (polysulfone) and lyophilized yielding 1.6 grams (20PLPEG550DAPEI4C18; lot#20080603b). Ten mg/ml of 20PLPEG550DAPEI4C18 was analyzed by GPC analysis using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 nm/min showed a retention time of 11.6 min or approx 21.4 nm molecular diameter. One mg/ml was analyzed for amino groups by TNBS and found to contain 76+/−5 uM NH2 or 76 mmol/mg.

### Example 12

Synthesis of 20PLPEG550DAPEI8C18 (Lot#2008604b): 20PLPEG550DAPEI8 (2 grams; lot#20080604a, see example 9 above) was dissolved in 5 ml of 1M HEPES with 4 ml of ethanol. Aliquot (4 ul) was taken and diluted to 4 ml for amino group measurement by TNBS and found to be 304 nmol NH2/mg. The 20PLPEG550DAPEI8 solution was warmed to 50 degrees Celsius and constantly stirred. Stearic acid or C18 fatty acid (196 mg; Mw=284.48; 0.69 mmol) was dissolved in 13 ml ethanol, 1.5 ml of 1M MES pH 4.7 was added followed by 82 mg NHS (Mw=115; 0.71 mmol), and 328 mg EDC (Mw=191.71; 1.71 mmol). Activation was allowed to proceed for 20 minutes. The activated C18 fatty acid was added to 20PLPEG550DAPEI4 solution and allowed to react for an hour. After an hour, the pH was adjusted to pH4.7 with 6N HCl and 328 mg EDC (mw-191.71; 1.71 mmol) was added. After 20 minute reaction, the pH was adjusted back to pH 7.1. The reaction mixture was cooled to room temperature and loaded into a 100,000 MWCO ultrafiltration cartridge (14FP-100-E-5A; GE-healthcare-Amersham), concentrated to 100 ml and washed with 10 volume changes of 80% ethanol, followed by 10 volume changes with water. Sample 20PLPEG550DAPEI8C18 was filtered sterilized using 0.2 um filter (polysulfone) and lyophilized yielding 2.1 grams (20PLPEG550DAPEI8C18; lot#20080604b). Ten mg/ml of 20PLPEG550DAPEI8C18 was analyzed by GPC using TosohG4000WXL column (0.79×30 cm) eluted with phosphate buffered saline (PBS; 11.9 mM phosphate, 137-M NaCl, 2.7 mMKPO₄, pH 7.4) containing 15% Acetonitrile at a flow rate of 0.6 ml/min. The retention time on GPC was found to be 11.6 min or approx 21.4 nm molecular diameter. One mg/ml was analyzed for amino groups by TNBS and found to contain 153+/−5 uM NH2 or 153 nmol/mg.

### Example 13

Synthesis of 20PLPEG550DAPEI12C18 (Lot#2008605b): 20PLPEG550DAPEI12 (2 grams; lot#20080605a, see example 10 above) was dissolved in 5 ml of 1 M HEPES with 4 ml of ethanol. Aliquot (4 ul) was taken and diluted to 4 ml for amino group measurement by TNBS and found to be 448 nmol NH2/mg. The 20PLPEG550DAPEI12 solution was warmed to 50 degrees Celsius and constantly stirred. Stearic acid or C18 fatty acid (289 mg; Mw=284.48; 1.0 mmol) was dissolved in 19 ml ethanol, 2.0 ml of 1M MES pH 4.7 was added followed by 120 mg NHS (Mw=115; 1.0 mmol), and 480 mg EDC (Mw=191.71; 2.5 mmol). Activation was allowed to proceed for 20 minutes. The activated C18 fatty acid was added to 20PLPEG550DAPEI12 solution and allowed to react for an hour. After an hour, the pH was adjusted to pH4.7 with 6N HCl and 480 mg EDC (mw=191.71; 1.71 mmol) was added. After 20 minute reaction, the pH was adjusted back to pH7.1. The reaction mixture was cooled to room temperature and loaded into a 100,000 MWCO ultrafiltration cartridge (UFP-100-E-5A; GE-healthcare-Amersham), concentrated to 100 ml and washed with 10 volume changes of 80% ethanol, followed by 10 volume changes with water. Sample 20PLPEG550DAPEI12C18 was filtered sterilized using 0.2 um filter (polysulfone) and lyophilized yielding 2.0 grams (20PLPEG550DAPEI12C18; lot#20080605b). Ten mg/ml of 20PLPEG550DAPEI12C18 was analyzed by GPC using TosohG4000WXL column (0.79×30 cm) eluted with phosphate buffered saline (PBS; 11.9 mM phosphate, 137 mM NaCl, 2.7mMKPO₄, pH 7.4) containing 15% Acetonitrile at a flow rate of 0.6 ml/min. The retention time on GPC was found to be 11.5 min or approx 22.2 nm molecular diameter. One mg/ml was analyzed for amino groups by TNBS and found to contain 206+/−5 uM NH2 or 206 nmol/mg.

### Example 14

Synthesis of 40PLPEG540PEI4C8: 1 g of 40PLPEG540-PEI-4 (0.4 mmol amino groups) was dissolved in 20 ml 75% ethanol 25% of 1 M HEPES and 2 ml (7.4 mmol) of n-Caprylic Anhydride was added. After 2 hours the amino group was measure by TNBS (Sparado et al. Anal Biochem 96:317, 1979) and found to be 0.1 mmol indicating 75% saturation of primary amino groups. This was washed with 10 volumes of ethanol and then 10 volumes of water using 100 kDa MW cutoff membrane and lyophilized (0.92 grams). This type of carrier, with hydrophobic groups, is designed to bind small negatively charged organic molecules such as prostaglandins.

### Example 15

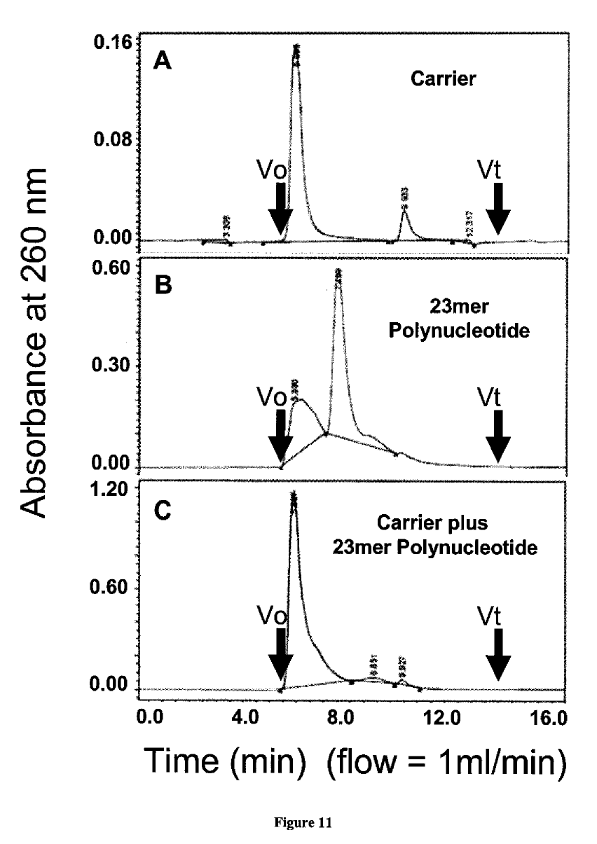

Binding of 23mer polynucleotide to the 40PLPEG540PEI4: 20 mg of carrier (40PLPEG540-PEI-4) was made up to 400 ul of water giving a 50 mg/ml solution of carrier. Polynucleotide (23 mer double stranded DNA with 2 nucleotide overhang) was made up to 2 mg/ml water. The sequence of representative polynucleotide used here is 5′-AAG AGA AGC GGC CAG TAT AGO TT-3′ and 5′-AA CCT ATA CTG GCC GCT TCT CTT-3′, where all bases are all deoxyribonucleotides. This was custom made by Midland Certified Reagent Company (Midland, Tex., USA) and any sequence can readily be made as the synthetic process is very well known in the art. The sequence of the representative polynucleotide sequence used here is 5′-AAG AGA AGC GGC CAG UAU AGG dTdT-3′ and 5′-Cholesterol-AA CCU AUA CUG GCC GCU UCU CdTdT-3′, where all nucleotides are ribo-nucleotides except those with a “d” representing “deoxyribo” such as dT. This was custom made by Biosynthesis (Lewisville, Tex., USA) and any sequence can readily be made as the synthetic process is very well known in the art. All these bind the 40PLPEG540-PEI-4 carrier in a way shown in FIG. 11. Carrier (2.5 mg/tube or 50 ul of 50 mg/ml carrier) was loaded with the 23 mer polynucleotide (0.20 mg/tube or 100 ul of 2 mg/ml of 23 mer polynucleotide) by placing them together in a tube, adding 25 ul of 10× phosphate buffered saline (10×PBS) and 75 ul water. These represent 100 ul of 2 mg/ml of 23 mer polynucleotide. Controls are a similar concentration of 23 mer polynucleotide in PBS and similar concentration of PBS. These were incubated for 2 hrs and several 50 ul aliquots of each were analyzed by SEC HPLC using Phenomenex BioSep-SEC-S-2000 at a flow rate of 1 ml/min in PBS (Results are shown in FIG. 11).

### Example 16

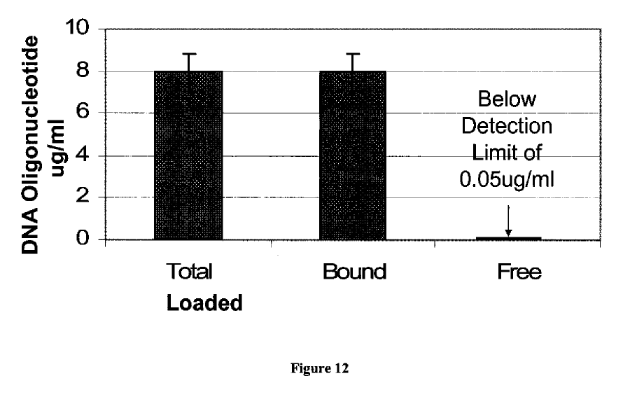

Binding of random dsDNA to the 40PLPEG540PEI12: 0.1 mg of carrier (40PLPEG540-PEI-12 from see example 5) was loaded with random DNA fragments (2 ug) and made up to 250 ul in PBS. Two preparations of random DNA alone (2 ug) in 250 ul PBS were made; one to be used as unfiltered control and the other to be used as filtered control. Similarly carrier (0.1 mg) in 250 ul PBS was used as a blank. All samples except the unfiltered control were filtered through 100 kDa molecular weight cut off filters (Microcon Ultracell YM-100, made up of regenerated cellulose from Millipore, Bedford, Mass.) by centrifugation at 12,000×g for 12 minutes. The filtrates were assayed for DNA using the DNA Assay kit (Quant-iT ds DNA from Invitrogen, Eugene, Oreg.) according to the manufacturer's instruction. The sample that went through the filter was the amount of free or unbound DNA as the carrier is too large to penetrate the filter. In the assay, the dsDNA binds fluorescent dye in the kit that fluoresces (excitation 485 nm n/emission 535 nm) at an intensity proportional to the amount of DNA in the sample. Unfiltered sample without carrier was assayed to represent the total DNA loaded and the result was found to be similar to the filtrate of the sample without carrier indicating that the filter does not retain DNA. The results showed that the carrier binds DNA very strongly, irrespective of the sequence. Because the DNA used is a random mixture of many different sequences, the results indicates that the binding is sequence independent and can be used for any polynucleotide independent of sequence (see FIG. 12).

### Example 17

GLP-1 Binding of 20PLPEG550DAPEIC18 Carrier: Incubation mixtures in triplicate were prepared to determine the ability of various carriers (20PLPEG550DAPEI4C18, 20PLPEG550DAPEI8C18, and 20PLPEG550DAPEI12C18; see above) to bind GLP-1. Before testing 20PLPEG550DAPEI4C18, 20PLPEG550DAPEI8C18, and 20PLPEG550DAPEI12C18 were supplemented with 0.15 umol, 0.15 umol, and 0.2 umol ZnCl/mg respectively. For 2% loading (wt GLP-1×100/wt of carrier), 250 ul test solutions were prepared in Phosphate buffered Saline (PBS; 11.9 mM phosphate, 137 mM NaCl, 2.7mMKPO₄, pH 7.4) containing 0.2 mg/ml GLP1, and 10 mg/ml Carrier. For 10% loading, 250 ul test solutions were prepared in Phosphate buffered Saline (PBS; 11.9 mM phosphate, 137 mM NaCl, 2.7 mMKPO₄, pH 7.4) containing 0.2 mg/ml GLP1, and 2 mg/ml Carrier. For 20% loading, 250 ul test solutions were prepared in phosphate buffered saline (PBS; 11.9 mM phosphate, 137 mM NaCl, 2.7 mMKPO₄, pH 7.4) containing 0.2 mg/ml GLP 1, and 1 mg/ml carrier. Control samples without carriers were also prepared. GLP-1 was obtained from ChemPrep (Miami, Fla.; lot#29306). The mixtures were vortexed and incubated for 2 hours. Samples and controls were filtered through 100 kDa molecular weight cut off centrifugal membrane filter (Ultracel YM-100; Millipore, Bedford, Mass.) by centrifugation at 14,000×g for 10 minutes. The filtrate was analyzed by reverse phase HPLC using Synergi 2.5 um Max-RP column (20×4 mm; 2.5 um; Phenomenex, Torrence, Calif.). Elution flow rate was 1.5 ml/min using gradient of solvent A and B as follows: 0.0-0.5 min 0.0% B-0.0% B; 0.5-1.0 min 0.0% B-25% B; 1.0-5.0 min 25%/B-50% B; 5.0-5.5 min 50% B-99%/B; 5.5-6.0 min 99% B-0.0% B; where solvent A is 5% Acetonitrile in water with 0.1% TFA and solvent B is 100% Acetonitrile with 0.1% TFA. The chromatogram was monitored at 220 nm and GLP-1 elutes at about 3.1 minutes and was quantified by determination of area under the peak. The area of the GLP-1 peak in the control samples were taken as 100% and it was established that that GLP-1 has negligible binding to the filter (regenerated cellulose filter used in Ultracel YM-100). The amount of free GLP-1 (that passed the filter into the filtrate) in the presence of various carriers (which did not pass the filter) as determined by HPLC is shown below (Table 1).

TABLE 1
(i) Binding of various cationic-core carriers containing C18 to GLP-1.

| | % Free (standard deviation: n = sample number) | | |
|---|---|---|---|
| Carrier Used | 20PLPEG555PEI4C18 | 20PLPEG555PEI4C18 | 20PLPEG555PEI4C18 |
| 2% Loading | 0% (0: n = 6) | 0% (0: n = 6) | 0% (0: n = 6) |
| 10% Loading | 0% (0: n = 6) | 0% (0: n = 6) | 0% (0: n = 6) |
| 20% Loading | 28.3% (5.2: n = 6) | 64.2% (3.4: n = 6) | 34.6% (1.2: n = 6) |

### Example 18

Insulin Binding of 20PLPEG550DAPEI Carriers: Incubation mixtures in triplicate were prepared to determine the ability of various carriers (20PLPEG1055DAPEI4; 20PLPEG1055DAPEI8; 20PLPEG550DAPEI4; 20PLPEG550DAPEI8; and 20PLPEG550DAPEI12 see above lot #20080415; 20080421; 20080603a; 20080604a; 20080605a) to bind insulin. Before testing 20PLPEG1055DAPEI4; 20PLPEG1055DAPEI8; 20PLPEG550DAPEI4; 20PLPEG550DAPEI8; and 20PLPEG550DAPEI12 were supplemented with 0.2 umol, 0.3 umol, 0.2 umol, 0.3 umol, and 0.4 umol ZnCl/mg respectively. For 2% loading (meaning: wt insulin×100/wt of carrier), 250 ul test solutions were prepared in AA buffer (100 mM Ammonium Acetate buffer, pH 7.4) containing 0.2 mg/ml Insulin, and 10 mg/ml carrier. For 5% loading (meaning: wt insulin×100/wt of carrier), 250 ul test solutions were prepared in AA buffer (100 mM Ammonium Acetate buffer, pH 7.4) containing 0.2 mg/ml Insulin, and 4 mg/ml carrier. For 15% loading (meaning: wt insulin×100/wt of carrier), 250 ul test solutions were prepared in AA buffer (100 mM Ammonium Acetate buffer, pH 7.4) containing 0.2 mg/ml Insulin, and 1.33 mg/ml Carrier. Control samples without carriers were also prepared. Insulin was a recombinant human insulin (Millipore Cat#4506 or NovoNordisk Product#306-8890; Millipore; Billerica, Mass., lot#TQ1HHP002). The mixtures were vortexed and incubated for 2 hours. Samples and controls were filtered through 100 kDa molecular weight cut off centrifugal membrane filter (Ultracel YM-100; Millipore, Bedford, Mass.) by centrifugation at 14,000×g for 10 minutes. The filtrate was analyzed by reverse phase HPLC using Synergi 2.5 um Max-RP column (20×4 mm; 2.5 um; Phenomenex, Torrence, Calif.). Elution flow rate was 1.5 ml/min using gradient of solvent A and B as follows: 0.0-0.5 min 0.0% B; 0.5-1.0 min 0.0% B-10% B; 1.0-5.0 min 10% B-50% B; 5.0-5.5 min 50% B-99% B; 5.5-6.0 min 99% B-0.0% B; where solvent A is 5% Acetonitrile in water with 0.1% TFA and solvent B is 100% Acetonitrile with 0.1% TFA. The chromatogram was monitored at 220 nm and insulin elutes at about 3.4 minutes and was quantified by determination of area under the peak. The area of the Insulin peak in the control samples were taken as 100% and it was established that that insulin has negligible binding to the filter (regenerated cellulose filter used in Ultracel YM-100). The amount of free Insulin (that passed the filter into the filtrate) in the presence of various carriers (which did not pass the filter) as determined by HPLC is shown below (Table 2).

### Example 19

Oligonucleotide (25-mer) Binding of 20PLPEG550DAPEI Carriers: Incubation mixtures were prepared in triplicate to determine the ability of various carriers (20PLPEG1055DAPEI4; 20PLPEG1055DAPEI8; 20PLPEG550DAPEI4; 20PLPEG550DAPEI8; and 20PLPEG550DAPEI12 see above lot #20080415; 20080421; 20080603a; 20080604a; 20080605a) to bind a 25-mer double stranded DNA fragment (Fermentas, Glen Burnie, Md., cat# sm1761). For 3.3% loading (meaning: wt of Oligonucleotide×100/wt of carrier), 50 ul test solutions were prepared in phosphate buffered saline (PBS; 11.9 mM phosphate, 137 mM NaCl, 2.7 mMKPO₄, pH 7.4) containing 13.2 ug/ml oligonucleotide, and 0.4 mg/ml Carrier). For 5% loading (meaning: wt of Oligonucleotide×100/wt of carrier), 50 ul test solutions were prepared in Phosphate buffered Saline (PBS; 11.9 mM phosphate, 137 mM NaCl, 2.7 mMKPO₄, pH 7.4) containing 13.2 ug/ml oligonucleotide, and 0.264 mg/ml Carrier. For 6% loading (meaning: wt of Oligonucleotide×100/wt of carrier), 50 ul test solutions were prepared in Phosphate buffered Saline (PBS; 11.9 mM phosphate, 137 mM NaCl, 2.7mMKPO₄, pH 7.4) containing 13.2 ug/ml oligonucleotide, and 0.22 mg/ml Carrier. For 6.6% loading (meaning: wt of Oligonucleotide×100/wt of carrier), 50 ul test solutions were prepared in Phosphate buffered Saline (PBS; 11.9 mM phosphate, 137 mM NaCl, 2.7 mMKPO₄, pH 7.4) containing 13.2 ug/ml oligonucleotide, and 0.20 mg/ml Carrier. For 7.4% loading (meaning: wt of Oligonucleotide×100/wt of carrier), 50 ul test solutions were prepared in Phosphate buffered Saline (PBS; 11.9 mM phosphate, 137 mM NaCl, 2.7 mMKPO₄, pH 7.4) containing 13.2 ug/ml oligonucleotide, and 0.179 mg/ml Carrier. For 12% loading (meaning: wt of Oligonucleotide×100/wt of carrier), 50 ul test solutions were prepared in Phosphate buffered Saline (PBS; 11.9 mM phosphate, 137 mM NaCl, 2.7 mMKPO₄, pH 7.4) containing 13.2 ug/ml oligonucleotide, and 0.110 mg/ml Carrier. For 13% loading (meaning: wt of Oligonucleotide×100/wt of carrier), 50 ul test solutions were prepared in Phosphate buffered Saline (PBS; 11.9 mM phosphate, 137 n-M NaCl, 2.7mMKPO₄, pH 7.4) containing 13.2 ug/ml oligonucleotide, and 0.102 mg/ml Carrier. For 15% loading (meaning: wt of Oligonucleotide×100/wt of carrier), 50 ul test solutions were prepared in Phosphate buffered Saline (PBS; 11.9 mM phosphate, 137 mM NaCl, 2.7 mMKPO₄, pH 7.4) containing 13.2 ug/ml oligonucleotide, and 0.088 mg/ml Carrier. For 30% loading (meaning: wt of Oligonucleotide×100/wt of carrier), 50 ul test solutions were prepared in Phosphate buffered Saline (PBS; 11.9 mM phosphate, 137 mM NaCl, 2.7mMKPO₄, pH 7.4) containing 13.2 ug/ml oligonucleotide, and 0.044 mg/ml Carrier. Control samples without carriers but only containing oligonucleotide (25-mer double stranded DNA fragment from Fermentas, Glen Burnie, Md., cat# sm1761) in the same buffer were prepared. The mixtures were vortexed and incubated for 2 hours. Samples and controls were filtered through 100 kDa molecular weight cut off centrifugal membrane filter (Ultracel YM-100; Millipore, Bedford, Mass.) by centrifugation at 14,000×g for 10 minutes. The filtrate was analyzed by ion exchange HPLC using LunaNH2-5 um column (30×4.6 mm; Phenomenex, Torrence, Calif.). Elution flow rate was 1.5 ml/min using gradient of solvent A and B as follows: 0.0-0.5 nm in 0.0% B; 0.5-1.0 min 0.0% B-25% B; 1.0-5.0 min 25% B-100% B; 5.0-5.5 min 100% B-0% B where solvent A is 20 mM Sodium Phosphate, 10% Acetonitrile, pH7.1 and solvent B is 20 mM Sodium Phosphate, 10% Acetonitrile, 2M Sodium Chloride pH7.0. The chromatogram was monitored at 260 nm and 25-mer oligonucleotide comes out 3.4 minutes and was quantified by determination of area under the peak. The area of the 25-mer oligonucleotide peak in the control samples was taken as 100% and it was established that the 25-mer oligonucleotide has negligible binding to the filter (regenerated cellulose filter used in Ultracel YM-100). The amount of free 25-mer oligonucleotide (that passed the filter into the filtrate) in the presence of various carriers (which did not pass the filter) was also determined. The data presented in Table 2 (% Free DNA@x % load) below is the percent free oligonucleotide over the total nucleotide used in the incubation mixture (% Free) at specific % loading (x % load) where the % load means the total nucleotide weight used in the incubation mixture is expressed as percent of total carrier weight used in the incubation mixture.

TABLE 2
(ii) Cationic-core carriers and their properties

| Lot | | Size | NH₂/mg | % Free Insulin | % Free DNA |
|---|---|---|---|---|---|
| numbers | Structure name | (nm) | (nmol) | @x % load | @x % load |
| 20070926 | 40PLPEG537PEI12 | 21 | 553 | ND | ND |
| 20071005 | 40PLPEG537PEI4 | 19 | 395 | ND | ND |
| 20080415 | 20PLPEG1055DAPEI4 | 23.1 | 186 | 2@5%; 2@15% | 0@3%; 5@5%; |
| | | | | | 13@6%; 17@12% |
| 20080421 | 20PLPEG1055DAPEI8 | 24.8 | 296 | 7@5%; 4@15% | ND |
| 20080603a | 20PLPEG550DAPEI4 | 20.6 | 204 | 3@2% | 0@3.3%; 5@5%; |
| | | | | | 4@6.6%; 18@13% |
| 20080604a | 20PLPEG550DAPEI8 | 22.2 | 304 | 11@2% | Jelly |
| 20080605a | 20PLPEG550DAPEI12 | 22.2 | 448 | 28@2% | 0@7.4%; 0@11%; |
| | | | | | 0@15%; 12@30% |
| 20080603b | 20PLPEG550DAPEI4C18 | 21.4 | 76 | ND | ND |
| 20080604b | 20PLPEG550DAPEI8C18 | 21.4 | 153 | ND | ND |
| 20080605b | 20PLPEG550DAPEI12C18 | 22.2 | 206 | ND | ND |

### Example 20

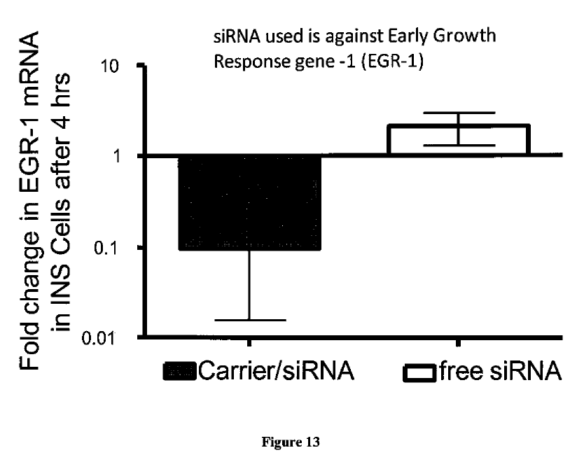

Cationic-core carrier protects siRNA and allows siRNA to silence mRNA in INS cell culture: INS cells (20,000 cells per well) were seeded into flat bottom 96well plate in 100 ul medium (RPMI; 10% FBS; 11.1 nM glucose; 10 mM pH 7.4 HEPES buffer; 1 mM sodium; 50 uM 2-mercaptoethanol) and let adhere over night in the incubator at 37° C. and 5% CO₂ atmosphere. Next day, the medium in each well was removed and fresh medium (10 ul) with or without appropriate amount of siRNA or siRNA-carrier complex were added and replaced back into the incubator. Corresponding siRNA against EGR-1 has the following sequence 5′AACCUAUACUGGCCGCUUCUCdTdT3′ and 5′AAGAGAAGCGGCCAGUAUAGGdTdT3′. After 4 hours, medium was removed and each well was rinsed with 200 ul ice cold PBS followed by addition of 50 ul of lysis buffer containing DNase (Taqman Gene Expression Cells-to-CT Kit; Applied Biosystems Cat#AM1728). The wells were mixed by pipetting up and down 5 times and incubated for 5 min at RT (19-25° C.). Five ul of stop solution (included in the kit) were added into each well, mixed by pipetting up and down 5 times and incubated for 2 min at RT (19-25° C.). The resulting RNA samples were frozen at −80 for further processing (cDNA first strand synthesis) the next day. For the first stand cDNA synthesis from the mRNA template, 40 ul of the RT Master mix (25 ul RT buffer, 2.5 ul RT Enzyme mix, and 12.5 ul nuclease free water per reaction: For 175 reactions: 4375 ul RT buffer, 437.5 ul RT Enzyme mix, and 2187.5 ul water) was added to the lysate RNA and incubated at 37° C. for 60 min, followed by 5 min 95° C., then put on ice. A portion of these cDNA samples (4 ul) were used for PCR and the remainder were frozen at −80° C. For PCR, 4 ul cDNA samples or nuclease free water (serving as a no template control) were placed into PCR tubes. Sixteen ul of a PCR cocktail, which is part of the kit (for a 20 ul reaction volume: 4 ul water, 10 ul TaqMan universal master mix, 1 ul EGR-1 primer/probe set that comes as a kit from Applied Biosystems Cat#Mm00656724-ml, 1 ul GAPDH primer/probe set that comes as a kit from Applied Biosystems Cat#4352339E per reaction), was added to the cDNA samples in PCR tubes, mixed gently by vortexing, centrifuge to collect content to the bottom, and transfer to real time PCR instrument (Applied Biosystems 7700 machine from Applied Biosystems) and run with default TaqMan settings (Hold at 50° C. for 2 nm in, hold at 95° C. for 10 min, and 40 cycles of 95° C. for 15 sec and 60° C. for 1 min). The GAPDH primer/probe set has a probe with a VIC fluorescent dye and recognizes the exon 3 location of the GAPDH gene and the EGR-1 primer/probe has a probe with a FAM fluorescent dye and recognizes the exon 2 and 3 locations of EGR-1 gene. The fluorescent dye in the probe is quenched by TAMRA dye which is released during amplification giving fluorescence proportional to the amount of amplicon. The primer pair for EGR-1 amplifies 182 bases in exon 2 and 3 of EGR-1 gene from the cDNA. The primer pair for GAPDH amplifies 107 bases in exon 3 of GAPDH gene from the cDNA. The TaqMan Gene Expression Master mix contains ROX™ passive reference dye. Positive results from a TaqMan PCR are visualized by at least two means: the amplification plot reflects the generation of the reporter dye during amplification and is related directly to the formation of PCR products. The intersection between the amplification plot and the threshold is defined as the cycle threshold, or CT, value. The CT value is related directly to the amount of PCR product and, therefore, related to the original amount of target present in the PCR reaction. The intersection between the amplification plot and the threshold, where the threshold is defined as 10 times the standard deviation of the background fluorescence intensity and which is measured between cycle 3 and 15, is known as the cycle threshold, or CT, value (default settings of the SD software may be changed manually). The CT value is directly related to the amount of PCR product and therefore related to the initial amount of target DNA present in the PCR reaction. The formula used to calculate fold change as shown in FIG. 13 is as follows:

### Fold Change=2^{-x}

Where X=[(CTEGR-1−CTGAPDH)]treatment −[(CTEOR-1−CTGAPDH)]No treatment
CT is cycle threshold corresponding to gene that follows it i.e. EGR-1 and GPDH;
Treatment indicates that the result is from cells treated with siRNA against EGR-1.
No treatment indicates that the result is from cells not treated with siRNA against EGR-1.

### Example 21

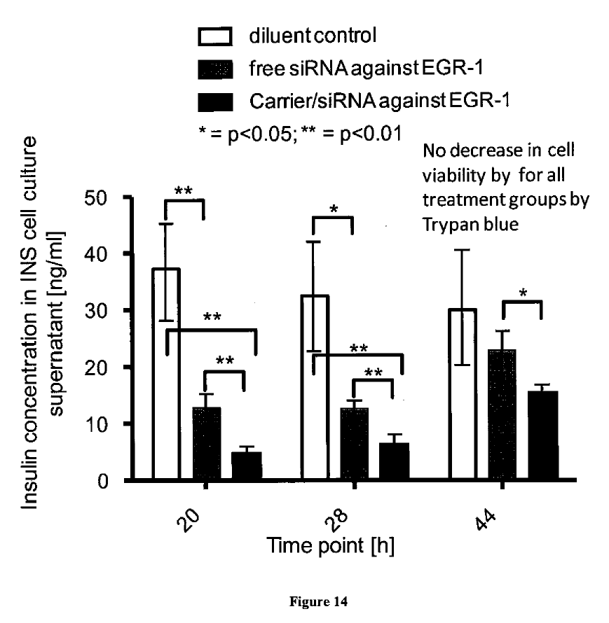

Decrease in insulin concentration in INS cell culture supernatant after treatment with siRNA: Insulin gene expression is indirectly controlled by EGR-1 mRNA level in INS cells through pancreas duodenum homeobox-1 (PDX-1) expression (Eto, K et al. (2006) Endocrinology, 147:2923-2935). Thus efficacy of siRNA against EGR-1 gene can be indirectly observed by decrease in insulin production. A decrease in insulin secretion 24 to 48 hours after EGR-1 mRNA destruction will provide a secondary evidence of siRNA effectiveness against EGR-1 and the ability of the carrier of the present invention to protect siRNA from serum degradation thus making it available to shut down EGR-1 mRNA in INS cells. INS cells were culture using RPMI (HyClone SH30027.01 lot# ASK 30524) medium containing 10% FBS (HyClone SH30088.03, lot # ARK27728), 1.1 mM glucose (Fisher), 10 mM HEPES (Fisher, BP299-100, lot#066175), 1 mM Sodium Pyruvate (HyClone SH30239.01, lot#ASB28758),50 uM b-mercaptoethanol (Fisher). INS cells (5×10⁶ INS-1 cells) were thawed, seed into 25 ml medium in 75 cm², and incubated at 37° C. and 5% CO₂ in humidified incubator. The medium was changed after 24 h and used only 115 ml for 75 cm² flask. Cells were seeded (105 cells/well) in a flat-bottom 96 well plate in 100 ul medium. The siRNA samples to be tested was prepared by dissolving 1.4 mg sense anti-EGR-1 RNA in 600 ul water and added to 1.4 mg anti anti-EGR-1 RNA followed by heating to 80° C. for 1 min and put on ice. An aliquot (300 ul or 300 uM) of this was added to 14 mg carrier (40PLPEG540PEI4; lot#20071005) giving 10% siRNA loading. The remaining 300 ul was used as the unformulated siRNA. These siRNA preparations were incubated on ice for 2 hours before use or before storing at −80 degree Celsius. The siRNA samples were diluted with INS-1 medium to 1000, 500, 100, 50, 10, 5, 1 and 0 nM of siRNA (700 ul each). The medium from 96well plate with cells was replaced 200 ul RNA/medium containing various concentrations of siRNA including 0 uM siRNA negative control, mixed and incubated at 37° C. Medium or supernatant was removed at 20, 28, and 44 hour for insulin analysis. At the end of the experiment, the cells in each well were treated with trypsin (Fisher, Cat#BW17-161F; 0.05% trypsin with 0.02% EDTA in saline) for 5 minutes and an aliquot was placed on a slide with grids appropriate for quantification and counted to correct for the amount of insulin in the medium. The amount of insulin in the medium (ng/ml) was normalized to 100,000 cells/well. Insulin was assayed using a commercially available Elisa Kit (Cat#EZRMI-13K, Linco, St. Charles, Mo.). Elisa plates come pre-coated with mouse anti-rat insulin antibodies where insulin in the sample can be captured on the surface of the Elisa plate wells. The secondary antibody is a biotinylated anti-insulin antibody and the reporter enzyme is a streptavidin-horseradish peroxidase conjugate. The reporter substrate is tetramethylbenzidine. For the reference curve, the absorbance at 590 nm is subtracted from absorbance at 450 nm and this difference is plotted on the y-axis against concentration of rat insulin standard on the x-axis. Concentration of insulin in the unknown sample was determined from this curve or from the equation derived from this curve. Results showing the effect of carrier on siRNA efficacy was seen through suppression of insulin production and shown in FIG. 14.

When ranges are used herein for physical properties, such as molecular weight, or chemical properties, such as chemical formulae, all combinations and subcombinations of ranges and specific embodiments therein are intended to be included.

From the foregoing it will be appreciated that, although specific embodiments have been described herein for purposes of illustration, various modifications may be made. Those skilled in the art will recognize, or be able to ascertain, using no more than routine experimentation, many equivalents to the specific embodiments described herein. Such equivalents are intended to be encompassed by the following claims. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

We claim:

1. A cationic-core carrier composition comprising: (i) a polymeric backbone; (ii) a plurality of polymeric protective chains covalently linked and pendant to the polymeric backbone; and (iii) a plurality of poly-cationic moieties covalently linked and pendant to the polymeric backbone, each with molecular weight of no more than 25% of the average molecular weight of the protective chains, wherein each protective side chain has a molecular weight between about 400 and 20,000 Daltons.

2. The composition of claim 1, further comprising a load molecule dissociably linked to the poly-cationic moiety.

3. The composition of claim 2, wherein the protective side chain is methoxypolyethylene glycol.

4. The composition of claim 3, wherein the polymeric backbone is polylysine.

5. The composition of claim 4, wherein the poly-cationic moiety is polyethyleneimine.

6. The composition of claim 5, wherein the load molecule is a therapeutic agent.

7. The composition of claim 6, wherein the therapeutic agent is agent is selected from the group consisting of a polynucleotide, an anionic peptide, an anionic protein, an anionic drug, and an oligonucleotide covalently bonded to a peptide or protein.

8. The composition of claim 6, wherein the therapeutic agent is a RNA or DNA polynucleotide.

9. The composition of claim 8, wherein the RNA polynucleotide is siRNA.

10. The composition of claim 9, wherein the siRNA is against any one from the group consisting of SSB gene, Ghrelin, NPY, Cathepsin, Myostatin, TSLP, IL-4, IL-8, L-12, IL-13, STAT-6, MIP-1 alpha, RANTES, CCR1, CCR3, INF-gamma, TNF-alpha, ICXCR1, MCP-1, and CCR2, CXCR3, CXCR4, CXCR5, CCR4, CCR5, CCR6, CCR7, CCR8, gp120, gp41, p17, p24, RT, HIV proteases, Fas (CD95), FAS-L, FADD, Caspase-8, IL-1, IL-6, Bak, Bax, Bid, Bcl-2, Bcl-XL, HLA-G, IGF-1, EGF, FGF, VEGF, VEGFR, IGFR, EGFR, FGFR, HER2, TGF-beta, Caspase 3, CEACAM6, HPV-E6, HPV-E7, H-Ras gene, P100a gene, CREB, BRAF gene, ATF2, N-myc gene, Cox1, Cox2, GluR2, DAT, VEGFR1, TGF-b-RII, IL-1-beta, Facipain-1-2 malaria protein, viral Capsid protein, NS5A, NP influenza protein, PA influenza protein, and HBV.

11. The composition of claim 9, wherein the siRNA is against VEGF.

12. The composition of claim 2, wherein the protective side chain is polyethylene glycol, polypropylene glycol, a co-polymer of polyethylene glycol and polypropylene glycol, polysaccharide, or alkoxy derivatives thereof.

13. The composition of claim 12, wherein the alkoxy derivative is methoxypolyethylene glycol, methoxypolypropylene glycol, a methoxylated co-polymer polyethylene glycol and polypropyleneglycol, or ethoxylated polysaccharide.

14. The composition of claim 2, wherein the polymeric backbone is selected from the group consisting of polylysine, polyaspartic acid, polyglutamic acid, polyserine, polythreonine, polycysteine, polyglycerol, polyhistidine, natural saccharides, aminated polysaccharides, aminated oligosaccharides, polyamidoamine, polyacrylic acids, polyalcohols, sulfonated polysaccharides, sulfonated oligosaccharides, carboxylated polysaccharides, carboxylated oligosaccharides, aminocarboxylated polysaccharides, aminocarboxylated oligosaccharides, carboxymethylated polysaccharides, and carboxymethylated oligosaccharides.

15. The composition of claim 2, wherein the linear polymeric backbone is polylysine.

16. The composition of claim 2, wherein the poly-cationic molecule is selected from polyethyleneimine, spermidine, spermine, putrescine, cadaverine, polylysine, poly-arginine, and derivatives thereof.

17. The composition of claim 2, wherein the poly-cationic moiety is polyethyleneimine.

18. The composition of claim 2, wherein the load molecule is a therapeutic agent selected from the group consisting of polynucleotides, anionic peptides, anionic proteins, and oligonucleotides covalently bonded to a peptide or protein.

19. The composition of claim 2, wherein the load molecule is a therapeutic siRNA.

20. The composition of claim 2, further comprising a targeting molecule covalently linked to the protective side chains.

21. The composition of claim 20, wherein the targeting molecule is selected from a group consisting of an antibody, fragment of an antibody, chimeric antibody, lectins, receptor ligands, proteins, enzymes, peptides, saccharides, quasi substrates of enzymes, cell-surface-binding compounds, and extracellular-matrix-binding compounds.

22. The composition of claim 2, further comprising hydrophobic groups covalently linked to the poly-cationic moiety, wherein each hydrophobic group has molecular weight of 15-700 Da, independent of the poly-cationic moiety.

23. The composition of claim 22, wherein the hydrophobic group is an alkyl group with 1 to 36 carbon atoms.

24. The composition of claim 22, or 23, wherein the load molecule is a prostaglandin.

25. The composition of claim 2, further comprising a second set of protective chains covalently linked to the poly-cationic moiety.

26. The composition of claim 15 wherein the protective chains linked to the polylysine are between 15% and 70% of the total amino acid residues on the polylysine.

27. The composition of claim 15 wherein the polymeric protective chains linked to polylysine are between 35% and 55% of the total amino acid residues on the polylysine.

28. The composition of claim 1 wherein the polymeric backbone is linear.

29. A cationic-core carrier composition comprising: (i) a polymeric backbone; (ii) a plurality of polymeric protective chains covalently linked and pendant to the polymeric backbone; (iii) a plurality of poly-cationic moieties covalently linked and pendant to the polymeric backbone; and (iv) a load molecule dissociably linked to at least one poly-cationic moiety.

30. A pharmaceutical composition comprising anyone composition selected from compositions in claims 2-29.

31. A method of delivering a load molecule to a subject comprising loading the molecule onto a composition of claim 1 and administering the composition to said subject.

32. A method of delivering a load molecule to a subject comprising administering a composition of claim 2 to the subject.


SEQUENCE LISTING (4 sequences)

SEQ ID NO: 1 - DNA, 23 bp, Artificial Sequence
  Description of Artificial Sequence Synthetic oligonucleotide
aagagaagcggccagtataggtt

SEQ ID NO: 2 - DNA, 23 bp, Artificial Sequence
  Description of Artificial Sequence Synthetic oligonucleotide
aacctatactggccgcttctctt

SEQ ID NO: 3 - DNA, 23 bp, Artificial Sequence
  Description of Artificial Sequence Synthetic oligonucleotide
aagagaagcggccaguauaggtt

SEQ ID NO: 4 - DNA, 23 bp, Artificial Sequence
  Description of Artificial Sequence Synthetic oligonucleotide
aaccuauacuggccgcuucuctt